(19) United States
(12) Patent Application Publication
(10) Pub. No.: US 2009/0181361 A1
(43) Pub. Date: Jul. 16, 2009

(54) Rapid test for detecting infection

(76) Inventors: Weidong Xu, Tampa, FL (US); Shyam Mohapatra, Lutz, FL (US); Arun Kumar, Wesley Chapel, FL (US)

Correspondence Address: CHRISTOPHER PARADIES, PH.D., FOWLER WHITE BOGGS BANKER, P.A., 501 E KENNEDY BLVD, STE. 1900, TAMPA, FL, 33602, US

(21) Appl. No.: 12/008,861

(22) Filed: Jan. 14, 2008

(51) Int. Cl. C12Q 1/70; G01N 33/53

(52) U.S. Cl. 435/5; 435/7.2

(57) ABSTRACT

A rapid test kit has a cellulose filter paper with a flow rate selected in a range of about 0.04 to about 0.4 ml/min/cm², such that rapid screening for disease or other conditions amendable to detection of antibodies or antigens may be made using bodily fluids, such as blood, serum and plasma. In one example of a process for using a rapid test kit, a diluting buffer dilutes a sample of a bodily fluid and is presented directly on a test area of the test kit, then a staining reagent, such as protein A conjugated with colloidal gold or another chromophore is added to the test area, and a destaining buffer is added to improve background contrast. By combining antigens capable of detecting antibodies of infectious and/or non-infectious agents, the rapid test has improved specificity and sensitivity compared to other tests, while using whole blood, serum and plasma in less than two minutes. Furthermore, the use of whole blood, without diminishing sensitivity, provides for a test procedure capable of being used in the field and in doctor's offices as a simple, inexpensive screening test.

### FIELD OF THE INVENTION

The field is test kits providing rapid detection and diagnosis of an infectious agent in a volume of fluid containing enough antibodies for detection of antibodies by the test kit.

### BACKGROUND

Many diseases are first diagnosed using screening tests and are confirmed by additional testing. It is known that screening tests must possess a high degree of sensitivity, whereas confirmatory assays must possess a high degree of specificity. Tests with high sensitivity are known to produce few false-negative results, whereas tests with high specificity produce few false-positive results. It is difficult to produce a test kit having both high sensitivity and a high degree of specificity. Those knowledgeable in the field recognize that a single kit for use in a field, home environment, or a doctor's office cannot meet both sensitivity and specificity in a rapid assay for diseases diagnosed by testing for viral and bacterial antibodies, such as antibodies for AIDS (e.g., HIV), tuberculosis, malaria, and hepatitis, for example. Instead, field tests are used only for screening and more specific tests are conducted in a controlled laboratory environment.

Blood may be stored for 7-14 days in order to screen for a virus, increasing risks for anaphylactic reactions, increasing potassium concentration, and decreasing its oxygen carrying capacity. There is a longstanding need for agencies to conduct local blood tests to screen donors. The ability to screen bodily fluids, such as blood, saliva and urine, using reliable and rapid test kits is an unfilled and longstanding need.

The most common screening test is the enzyme-linked immunosorbent assay (ELISA), sometimes called enzyme immunoassay (EIA). The most often used confirmatory test is the Western blot. If antibodies are being produced in the body, these tests are capable of detecting the antibodies at low levels. For example, the conventional HIV testing protocol starts with a sensitive EIA in a clinical laboratory. The EIA might be performed with serum, plasma, urine, or oral fluid, and the results might be available in 3 to 4 days. If the EIA is negative, the result is considered definitive, and no further testing is indicated.

A limitation of any testing is that many viral antibodies take up to 3 months to express after infection occurs, causing a window between the infection and detection using even the most sensitive of assays. If the EIA is repeatedly positive, more specific testing, using the Western blot technique, is done for confirmation. The testing process from the time a specimen is submitted until a final result is available is often a week or even longer. The cost and time required to complete a test make frequent testing, even among high risks groups, impractical.

The Western blot test (WB) uses an electrical field that separates out the various components of a sample by molecular weight. This allows identification of antibodies to specific viral antigens, which show up as identifiable “bands” on a strip of test paper. This test offers a high degree of specificity. ELISA combines the specificity of antibodies with the sensitivity of simple enzyme assays, by using antibodies or antigens coupled to an easily assayed enzyme that possesses a high turnover number. ELISA can provide a useful measurement of antigen or antibody concentration, which is unavailable in rapid test kits. Herein, a rapid test is one that provides for a buffered specimen of blood or serum to be used in a test requiring less than five minutes to complete.

Whereas ELISA measures an antibody to a whole virus and gives a “positive,” “negative” or indeterminate test result, Western blotting is a more specific test. It allows one to visualize antibodies directed against each viral protein. For this reason, it is a confirmatory test for a positive test done with ELISA or EIA. The Western blot test is considered a gold standard test for the confirmation of an ELISA and/or a rapid assay screened reactive sample in the diagnosis of many viral infections, especially in the low risk population. Essentially, any repeatedly positive result by ELISA or another rapid screening method for many viral infections must be confirmed by a more specific assay such as a Western blot (WB) test.

This strategy of using both ELISA and supplementary tests further increases the accuracy of results and diagnosis. In principle, any WB kit that gives a high frequency of indeterminate reactivity (the overwhelming preponderance of which represents non-specific binding) is not appropriate as a primary screening tool for the population at large. Its strength is only as a confirmatory assay in the setting of a positive or indeterminate viral antibody ELISA or initial screening test.

In one example, the window period is the period between the onset of viral infection and the appearance of detectable antibodies to the virus. In the case of the most sensitive HIV antibody tests currently recommended, the window period is about three to four weeks. This period can, however, be longer. Any antibody-based blood test (such as the ELISA, rapid tests and the Western blot) conducted during this window period may give false negative results. The expense and time that these tests take means that testing is conducted infrequently on individuals. Although the virus is present in the person's blood there may be no (detectable) antibodies in the blood during a screening test for a period up to about three months, but the cost of testing increases this window to a year or more, especially if the individual is in a low risk group. Indeed, the onset of symptoms of disease is often the first indication in most patients. Waiting until the onset of symptoms of disease has the potential of exposing others to disease and dramatically diminishes the ability to treat a patient, in most cases. This is true for AIDS, hepatitis, tuberculosis and many other diseases that are proving increasingly difficult to treat, at least for some strains, with conventional antiviral or antibiotic regimens. During this window period and until a subsequent test is performed, the individual is already infectious and may unknowingly infect other people. What is needed is a rapid, inexpensive and sensitive test for detecting infectious diseases that permits routine testing of individuals at office visits, testing sites, blood donation centers, or even at home.

There has been an increase in the number of test kits for detecting infectious agents, such as viral and bacterial diseases. Unfortunately, there are many examples of test kits marketed for home use that are neither approved nor adequately tested for diseases such as AIDS. The only approved test kit for HIV in the United States takes a sample and sends the sample to a laboratory for analysis. No known rapid test kits that do not require sending a sample to a laboratory are approved for use in screening for HIV in the United States.

Some test kits are available for testing serum samples for disease. For example, test kits are available that include lateral flow tests. Lateral flow tests, also called immunochromatographic strip tests, are used for specific screening or semi-quantitative detection of many analytes including antigens and antibodies. Samples may either be used alone or with an extraction reagent, or running buffer, which is then placed on a sample pad on one end of a test strip. The test strip also includes a membrane. A signal reagent, is solubilized and binds to an antigen if present in the sample and moves through the membrane by capillary action. The complex is then captured by a second antibody, which produces a visible line, indicating presence of the antigen. Lateral flow tests are slow, but contrast is improved between the visible line and the background compared to directly depositing the sample in the test area. For this reason, lateral flow tests dominate the market for enzymatic testing of bodily fluids. No lateral test is known that is capable of using blood.

Flow through tests may involve kits as individual cassettes with extraction and wash buffers included. These tests involve capturing of an analyte such as antibody or an antigen by a reagent as it flows through a membrane. These test kits often suffer from poor contrast. The protocols may require a user to prepare the sample to be tested, to wash the membrane, to add a signal reagent, and to wash the membrane to clear the membrane of any residue from the sample in an attempt to improve the contrast between the background and any screening line or marker for indicating the presence of an enzyme or antibody. Direct, flow-through test kits are known to be rapid but are seldom used in practice due to the complexity of the protocol required to provide enough contrast between the indicator and the background membrane. It is known in the field that lack of contrast makes flow through test kits less sensitive than lateral flow test kits, and this teaches away from the use of flow through test kits. Complicated instructions for washing and rewashing the kits makes results, in practice, less consistent than results for a lateral flow test kit, which also mitigates against flow-through tests. Examples of testing with several other commercial tests kits are reported and compared here with examples of the present invention using antigens for detecting HIV antibodies. None of the commercial samples tested with whole blood worked, which limits the usefulness of any of these commercial test kits for field use where a laboratory and centrifuge are not available. Also, some examples had better contrast than commercial test kits, which makes them much easier to read.

Chen in WO 96/21863, describes an immunoassay test device for detection of antibodies to HIV-1 and HIV-2 in biological fluid, providing for immediate immunoreaction and detection of the presence of such antibodies, comprising an assembled filter device and reaction cell using a nitrocellulose membrane on which an immunoreaction occurs. Visualizing the antibodies that react with HIV antigenic glycoproteins gp41, gp36, gp38 and gp120 occurs by conjugating the antibodies with a Protein A colloidal indicator and viewing the membrane for the presence of a red color, indicating the presence of antibodies. Chen teaches a lateral flow and/or filtering of blood through a filtration medium before contacting a nitrocellulose membrane. The extra step of filtration first before contacting the membrane increases the time required for performing the test. Chen, in another publication, WO 95/18624, teaches a similar device that requires a nitrocellulose membrane. In this test, Chen uses only one protein, gp41. Western blot tests require presence of two of three HIV proteins for improved specificity; however, increasing the number of proteins detected does not reliably lead to improved sensitivity and specificity. In some cases, Western blot provides an indeterminate result that may actually indicate a specimen positive for HIV. Abbott Determine™ is an early screening test for HIV 1 and 2, but it does not provide a rapid test kit capable of use in the field with whole blood, for example.

Mahajan, in US Patent Publication No. 2004/0023210, discloses a diagnostic kit for detection of antibodies of Hepatitis C virus in human serum and plasma, which comprises a base, an immunofiltration membrane of nitrocellulose mounted over an absorbent pad disposed on the base, and a top cover removably attached to the base having a central hole conforming to the membrane's circumference. Antigens such as NS3, NS4, and NS5 are immobilized on the membrane and visualized with a Protein A conjugate. This reference teaches that the pore size of the nitrocellulose membrane is 0.8-1.5 microns. The pore size is poorly correlated with specificity and sensitivity, which are correlated with contrast (or color index values as reported herein). Test kits suitable only for use with serum or plasma are not suitable for use as rapid field test kits.

Hu, in U.S. Patent Publication No. 2003/0165970, teaches a diagnostic device for simultaneously detecting multiple infectious agents, such as HIV antibodies, Hepatitis B and C antibodies and syphilis antibody. The kit disclosed by Hu comprises an immunogold filtration assay device, buffer and a mixture of colloidal gold particles where the device includes a nitrocellulose membrane blotted with HBsAg monoclonal antibody, HCV antigen, syphilitic antigen, HIV antigen, and goat anti-mouse IgG antibody. The test is not rapid and requires a very complicated protocol.

Chu, in U.S. Pat. No. 5,885,526, discloses a flow-through test device having a reaction membrane that includes porous material, such as nitrocellulose. A small pore size is taught to be needed when using nitrocellulose membranes in order to provide a greater area for immobilizing receptor molecules. Chu teaches that larger pore sizes lead to decreased assay sensitivity, as described in col. 5, Ins. 53-56. Chu prefers the porosity of the reaction membrane to be in a range from 0.45 to 3 microns. Chu teaches away from using compression to hold the reaction membrane, as it makes the device less suitable for some immunoassays where quantitative results are needed, as disclosed in col. 3, Ins. 15-32, and Chu fails to disclose any example using whole blood with cellulose filter papers.

The examples in Chu also teach away from increasing flow rate, which Chu describes as decreasing interaction time between a target molecule in the sample and an immobilized receptor on the reaction membrane. Thus, assay sensitivity decreases as disclosed in col. 5, Ins. 57-60. Again, pore size is a poor predictor of sensitivity and specificity.

Chu also teaches that a thick reaction membrane is needed to form an air pocket to prevent lateral flow and direct flow. The working example discloses a thick 800 micron paper-backed nitrocellulose reaction membrane, as disclosed in Example 2. Chu also discloses many disadvantages of prior art devices which have thin reaction membranes such as membranes being less than 0.1 mm thick, as disclosed in col. 7, Ins. 66-col. 8.

Chu, discloses that a membrane should be capable of immobilizing an antigen and Protein A and he suggests materials such as nitrocellulose and fiberglass as being suitable for immobilizing the antigen and Protein A. Chu requires an inoculation of both Protein A and an antigen at different areas of the membrane before testing of an analyte sample. Chu also requires both protein A and an antigen of interest to be inoculated on the membrane first before a serum sample is absorbed into the membrane and also utilizes an additional step of adding protein A-colloidal gold conjugate to be added after the serum or plasma is absorbed, which makes Chu's preferred protocol, which is necessary to provide adequate contrast, very complex and not at all rapid. In addition, Chu discloses inoculation of Protein A to be preferably at an edge of a device, as the central location of the membrane will contain an antigen of interest, such as a Hepatitis C antigen. Chu in another patent, U.S. Pat. No. 5,541,059, discloses an immunoassay device employing Protein A and an antigen. The test kits of Chu are not rapid test kits and suffer from complicated protocols, and unpredictable results in the hands of less trained staff and individuals. Chen et al., in U.S. Patent Publication No. 2004/0002063, prefers a porous reaction membrane such as paper-backed nitrocellulose, and a preferred pore size of 0.2 to 0.8 microns, as disclosed in paragraph [0062]. The membranes disclosed in Chen must be suitably porous membranes, such as the examples disclosed that use a nitrocellulose backed with porous paper. Testing of nitrocellulose membranes show that flow rate of water through the membranes are very rapid, but nitrocellulose failed in tests conducted by the applicant. While Chen does not exclude cellulose filter paper as a membrane, cellulose filter paper having a flow rate comparable to nitrocellulose is inoperable, as shown by the applicants results. No examples are provided by Chen using cellulose filter paper as a membrane in any test kit. Also, Gelman et al., in U.S. Pat. No. 5,980,746, teaches away from the use of cellulose compounds because it is well known in the art that cellulose compounds, “reduce membrane adsorbability of proteins,” for example. Thus, it is known to use nitrocellulose membranes in testing for the presence of antibodies.

In addition, for testing blood, Chen discloses that a more complex test kit having a separate blood separation zone is needed, such as one using a glass fiber matrix as the blood separation material, an example provided in paragraph [0089] of U.S. Patent Publication No. 2004/0002063, for example. This complicated procedure is not viable as a field test.

Krutzik, in U.S. Pat. No. 6,653,066, discloses a lateral flow test using a matrix pore size of less than 5 microns and nitrocellulose membranes and discourages the use of larger pore sizes, which tends to have poor results.

It is well-known to collect dried whole-blood spots on cellulose filter paper, but this is used for collection and drying of blood and not as a rapid test kit. Indeed, the characteristics that make cellulose filter papers attractive for storing blood are counterintuitive for test kits. The dried blood is later washed from the filter paper and is used for testing. For example, Rocks et al., in Ann. Clinical Biochemistry 1991; 28:155-159, describes collecting blood on filter paper before evaluating the samples in an antigen coated microtitration wells, using a silver-enhanced gold-labelled immunoassay. Patton et al., in Clinical and Vaccine Immunology, January 2006, pgs. 152-153, describes the use of filter paper for gathering blood samples for further analysis with HIV-1 p24 ELISA assay, but this does not use the cellulose filter paper as a membrane or in the test. Instead, the blood is washed from the filter paper and is used in a separate test. Fortes et al., in Journal of Clinical Microbiology, June 1989, pgs. 1380-1381, describes the use of a cotton-filter paper before conducting further ELISA tests. None of these references disclose any type of rapid test kit. Instead, the filter paper is used to transport and store dried blood, which is washed from the filter paper.

### SUMMARY OF THE INVENTION

A rapid test for detecting infection is capable of detecting HIV infection in less than five minutes. In some examples, test kits have comparatively low flow rates and large particle retention size (correlating with pore size) and are capable of completion of a rapid test in less than 3 minutes. In one example, a test kit uses an antigen or a combination of antigens immobilized on a cellulose filter paper, the filter paper being selected to have a flow rate in a range from about 0.04 ml/min/cm² to about 0.4 ml/min/cm². The test kit is capable of detecting antibodies by direct deposit, flow-through of a buffered suspension such as PBS buffered blood, serum or plasma, for example. None of the other tested commercial test kits were capable of testing whole blood, which was readily achieved using examples of the present invention without affecting the outcome and with similar contrast to the same test using serum or plasma.

In one example, a diagnostic kit includes an antigen-immobilizing cellulose filter paper, at least one antigen immobilized on the cellulose filter paper, a staining agent to detect antibodies against the at least one antigen, a destaining buffer to remove non-specific background staining, and a plurality of wicking layers disposed in a bottom portion of the diagnostic kit opposite of the reaction membrane. For example, a cellulose filter paper used as a reaction layer of the test kit may have a particle retention size selected in a range from about 6 to about 25 microns. Furthermore, test results for a variety of particle retention sizes for cellulose filter papers show that papers having particle retention sizes of 6, 11, and 20-25 (Whatman Qualitative/Wet Strengthened grade cellulose filter papers) do not exhibit a large departure in flow rate. A rapid test kit should not have a flow rate unnecessarily low, but there is a correlation between flow rate and a color index value reported in the results, which is related to sensitivity of the test kit for detecting antibodies. Thus, there is a preferred range for selecting cellulose filter paper with an optimum flow rate.

In one example, a staining agent is Protein A coupled to colloidal gold. A destaining buffer is used, such as phosphate buffered saline (PBS) to improve contrast with the background.

In another example, a rapid test for detecting infection selects cellulose filter paper or an equivalent that has a phosphate buffer saline (PBS) solution flow rate in a range between about 0.04 to about 0.4 ml/min/cm², more preferably 0.04 to 0.2 ml/min/cm² for higher contrast (sensitivity). Flow rate, is more important than pore size in determining assay sensitivity and time to complete the test. In one embodiment, a cellulose filter paper can be selected to have a flow rate in a range from about 0.1 to about 0.2 ml/min/cm², providing an optimum trade-off in sensitivity and flow rate for some examples.

In another example, the cellulose filter paper can be selected to have a PBS flow rate in a range from about 0.2±0.05 ml/min/cm² to increase flow rate without unduly sacrificing sensitivity (i.e., color index value). In flow rate measurements, the term “about” is used to indicate the manufacturing variances in manufacturing cellulose filter paper and in testing of flow rate according to the modified ASTM method described herein. A person of ordinary skill in the art will be able to measure flow rates and select cellulose filter papers based on the disclosed flow rate testing method and flow rates and those cellulose filter papers having about the same flow rates as the ranges given herein.

One advantage of the diagnostic kit using cellulose as a reaction layer is the ability to obtain rapid results for a particular infectious agent or a plurality of infectious agents without complicated user protocols. Indeed, results are provided as readily for whole blood as for serum or plasma in some examples.

Another advantage is the cost of a test kit, which substantially reduces the costs associated with screening. A rapid test kit is inexpensively produced and provided at low cost, which is especially necessary for use in remote locations and doctor's offices. A single test may be used to test more than one type of disease detectable from blood.

Yet another advantage is that a single diagnostic kit may be used in detecting one or more of a variety of bodily fluids, such as blood, plasma and serum, thus offering greater flexibility in testing. Field tests may be administered without the need of a mobile laboratory or a centrifuge.

Yet another advantage is that the rapid test kit provides a rapid result and both good sensitivity and good specificity.

### BRIEF DESCRIPTION OF THE FIGURES

The patent or application file contains at least one drawing executed in color. Copies of this patent or patent application publication with color drawing(s) will be provided by the Office upon request and payment of the necessary fee.

The drawings describe some examples of a rapid diagnostic kit and a method for preparing and using the diagnostic kit.

FIG. 1A illustrates an example of a cross section of a diagnostic kit 100.

FIG. 1B illustrates another example of a cross section of a diagnostic kit 110.

FIG. 1C depicts a top plan view of a diagnostic kit such as those shown in FIGS. 1A and 1B.

FIG. 2 (a) and (b) show top views of examples of test kits that (a) tested negative for the presence of an antibody and (b) tested positive for the presence of an antibody.

FIG. 3 (a) and (b) compare (b) an example of a diagnostic kit using a cellulose filter paper and (a) a glass fiber membrane, which resulted in failure when tested with blood.

FIG. 4A (1) and (2) compare examples of (2) an example of a diagnostic kit using a cellulose filter paper membrane and (1) a nitrocellulose membrane, which failed when tested with plasma.

FIG. 4B illustrates another examples using a nitrocellulose mixed ester membrane, which failed when tested with a diluted plasma sample.

FIG. 5 graphs color index value versus flow rate of PBS, as measured using a modified ASTM flow rate procedure with 7 cm circles of the cellulose filter papers used in the tests.

FIG. 6 illustrates a color index chart for determining color index values where any marker discernable over background is given a value of 1, anything darker than 1 is 2, anything darker than 2 is 3, and anything darker than 3 is deemed a 4, quantifying color intensity of test samples.

FIG. 7 shows measured flow rate versus particle retention size for 6 different cellulose filter papers.

FIG. 8 discloses a graph color index value by sample number for various results including testes using blood and plasma with a test kit having a PBS flow rate of about 0.1 mil/min/cm², and also showing color index of control spots.

FIG. 9 (a)-(d) compare the results of (a) & (b) blood and (c) & (d) plasma using otherwise similar test kits.

FIG. 10 graphically compares test results for plasma using a rapid test kit of the examples using a cellulose filter paper having a flow rate of about 0.1 ml/min/cm² and a commercially available test kit (Reveal® G3)¹. 1 Reveal® is a registered trademark of MedMira Laboratories, Inc., Toronto, Canada.

FIG. 11 (a)-(h) compare examples of results between (a)-(d) an example of a rapid test kit and (e)-(h) a commercially available test kit, for testing low titer samples.

FIG. 12 (a)-(d) compare blood samples using (a) a commercially available kit having a nitrocellulose membrane, (b) a rapid test kit with a cellulose membrane and (c), (d) a glass fiber membrane, for tests using blood.

FIG. 13 (a)-(f) compare plasma samples using (d)-(f) a rapid test kit using a cellulose filter paper and (a)-(c) a commercially available test kit, showing improved contrast of the rapid test kit using cellulose filter paper for the membrane.

FIG. 14 (a)-(f) provide additional comparisons between (a)-(c) a commercial test kit and (d)-(f) a rapid test kit using a cellulose membrane and having improved contrast.

### DETAILED DESCRIPTION

The examples described and the drawings rendered are illustrative and are not to be read as limiting the scope of the invention as it is defined by the appended claims. Additional advantages of the invention shall be apparent to a person of ordinary skill from the description of the examples provided.

A rapid diagnostic assay provides a quick and inexpensive screening test for detecting antibodies resulting from disease-causing organisms, such as a viruses, bacteria, fungus, mold and other disease-causing organisms that are detectable through an antibody assay. The diagnostic assay is a rapid assay meaning that the time to conduct the test from drawing of a bodily fluid to completing the test is rapid (e.g. less than ten minutes) and the time to obtain a test result after preparing a buffered suspension is rapid (e.g. less than one minute). Rapid test kits are not known that have both the sensitivity and the specificity of test kits used in the examples. Furthermore, none of the test kits known to the inventors are able to provide a result in less than 1 minute from the time that PBS buffered samples are ready to be used, such as shown for test kits obtaining strong positives in high titer tests and excellent results in low titer tests, also. Rapid is meant to mean both time scales (test preparation to completion and time for the test kit to provide a result after the test sample is mixed in buffer solution). Furthermore, examples of test kits provide rapid diagnostic assay using whole blood, serum or plasma as testing material. Whole blood is particularly problematic for all of the commercial test kits tested.

In one example, a method of rapid diagnostic assay uses the test kit of the examples in the field without any need of medical or laboratory facilities. Ability to distribute to remote locations makes testing convenient and inexpensive.

Various types of antigens may be used in a rapid diagnostic assay. The antibodies detected by a rapid diagnostic assay may be produced in response to bacteria, fungi, parasites, or viruses, for example. A wide variety of antigens may be used separately or together in a screening array. In addition to infectious diseases, the rapid diagnostic assay may also detect antibodies or antigens in non-infectious diseases such as cancer, Alzheimer's disease, or other non-infectious diseases.

Bacterial antigens Bacterial pathogens may be detected by a rapid test kit. In one example, an antigen is selected from a major outer membrane protein within strains of the genus Actinobacillus. For example, the antigen is disclosed in U.S. Pat. No. 6,541,011. In another example, a bacterial antigen may be from any of the following: Actinomyces, such as an ornithine-rich antigen from Actinomyces naeslundii, or Actinomyces viscosus as disclosed in U.S. Pat. No. 6,974,700; Aerobacter aerogens or Actinomyces israelli; Bacillus, such as Bacillus anthracis or Bacillus cereus or Bacillus subtilis or a protective antigen, lethal factor or an edema factor of Bacillus anthracis, as disclosed in WO 2004/024067; cell surface antigens of B. cereus as disclosed in U.S. Pat. No. 6,699,679; a 69 Kd protein of B. pertussis as disclosed in U.S. Pat. No. 5,527,529; Bacteroides; Bordetella; B. pertussis, such as mentioned in U.S. Pat. No. 6,197,548; B. parapertussis and B. bronchiseptica with molecular masses of 70 and 68 kDa respectively; Bartonella; Borrelia, such as Borrelia recurrentis or OspA of the Lyme disease Borrelia burgdorferi, as mentioned in U.S. Pat. No. 6,541,011; Brucella, such as Brucella abortus or Brucella melitensis, such as Omp29 on Brucella melitensis as mentioned in U.S. Pat. No. 6,541,011 or Brucella suis; Campylobacter, such as Campylobacter pylori as mentioned in U.S. Pat. No. 5,549,051; Capnocytophaga; Chlamydia, such as Chlamydia traqchomatis or Chlamydia psittaci, such as 80-90 kDa protein and 110 kDa protein, chlamydial exoglycolipid (GLXA), Chlamydia pneumoniae species-specific antigens in the molecular weight ranges 92-98, 51-55, 43-46 and 31.5-33 kDa and genus-specific antigens in the ranges 12, 26 and 65-70 kDa, as mentioned in U.S. Pat. No. 6,541,011; Clostridium, such as Clostridium botulinum or Clostridium perfingens or Clostridium tetani or a C fragment from C. tetani as mentioned in U.S. Pat. No. 5,527,529; A, B, C, and D toxoids from C. perfringens, such as a B toxoid as mentioned in U.S. Pat. No. 6,524,592 or toxin A from C. difficile, as mentioned in U.S. Pat. No. 6,503,722 or LT and HT toxins from C. sordellii disclosed in U.S. Pat. No. 6,849,715 or an alpha toxin from C. septicum of U.S. Pat. No. 7,037,503 or A-G toxins from C. botulinum as disclosed in U.S. Pat. No. 6,613,329; Corynebacterium, such as Corynebacterium diptheria; Coxiella; Dermatophilus; Enterococcus; Ehrlichia; Echinococcus granulosus antigen 5, as disclosed in U.S. Pat. No. 6,541,011; Escherichia coli; Francisella; Fusobacteria; H. pylori, such H. pylori GroES homologue (HspA) and four immunoreaction proteins of 45-65 kDa, as mentioned in U.S. Pat. No. 6,541,011; Hemophilus influenzae; H. ducreyi; H. hemophilus; H. aegypticus; H. parainfluenzae; Haemobartonella; Helicobacter; Klebsiella, such as the K antigen of Klebsiella pneumonia, disclosed in U.S. Pat. No. 6,541,011; Leptospira icterohemorrhagiae, such as Leptospira canicola; Leishmania, such as gp63 of Leishmania major, disclosed in U.S. Pat. No. 6,541,011; mycobacterial heat shock protein 65, disclosed in U.S. Pat. No. 6,541,011; Leptospira; Listeria, such as Listeriolysin 0 of Listeria monocytogenes as disclosed in U.S. Pat. No. 5,830,702; Moraxella, such as the CD protein of Moraxella, mentioned in U.S. Pat. No. 6,541,011; Mycobacteria, such as Mycobacterium avium or Mycobacterium bovis or Mycobacterium leprae or Mycobacterium paratuberculosis or Mycobacterium tuberculosis hominis or a 35 kilodalton protein of Mycobacterium leprae, as disclosed in U.S. Pat. No. 6,541,011, or 85 or 45/47 kDa-antigen of Mycobacterium tuberculosis, as disclosed in U.S. Pat. No. 6,541,011 or 18-kilodalton protein of Mycobacterium lepraes, as disclosed in U.S. Pat. No. 6,541,011; Mycoplasma. In one example, the antigen is from Mycoplasma hominis. In one example, the antigen is from Mycoplasma pneumoniae.

In one example, the bacterial antigen is from Neisseria. In one example, the antigen is from Neisseria gonorrhea. In one example, the antigen is from Neisseria meningitidis. In one specific example, the antigen is Por, Rmp or a LOS protein of Neisseria gonorrhoeae. In another example, the antigen may include PorA, Por B, Rmp, Opc, FrpB, TbpB or Nsp may be used, as mentioned by U.S. Pat. No. 6,797,273; Neorickettsia; Nocardia; Pasteurella, such as Pasteurella pestis; Peptococcus, such as Peptostreptococcus; Pneumococcus, such as Diplococcus pneumonia; Proteus; Pseudomonas; P. gingivalis, such as the 43-kDa and the fimbrilin (41 kDa) proteins of P. gingivalis, as disclosed in U.S. Pat. No. 6,541,011; Rickettsia, such as Rickettsia australis or Rickettsia burneill or Rickettsia conori or Rickettsia mooseri or Rickettsia prowazekii or Rickettsia tsutsugamushi; Rochalimaea; Salmonella, such as Salmonella choleraesus or Salmonella typhimurium or Salmonella typhosa or O, H, and Vi antigens of Salmonella or SEF14 fibrial antigen of Salmonella enteriditis and flagellar (G) antigens observed on Salmonella enteritidis and S. pullorum, disclosed in U.S. Pat. No. 6,541,011; Shigella, such as Shigella arabinotardo or Shigella boydii or Shigella dysenteria or Shigella flexneri or Shigella schmitzii or Shigella sonnei or O-antigens disclosed by U.S. Pat. No. 5,958,686 or S. dysenteria, disclosed in U.S. Pat. No. 5,204,097; Staphylococcus, such as Staphylococcus aureus or Staphylococcus albus or type 5, type 336, type 4, K73 antigens of S. aureus, disclosed by U.S. Pat. No. 6,537,559; hyperimmune serum reactive antigen of S. epidermidis, as suggested by U.S. Patent Publication 2007/0036778; ORF-2 antigen of Staphylococcus aureus or GlpQ, each disclosed in U.S. Pat. No. 6,541,011; Streptococcus, such as Streptococcus agalactiae (Group B Streptococcus), Streptococcus (viridans group), Streptococcus faecalis, Streptococcus bovis, Streptococcus (anaerobic sps.), or Streptococcus pneumoniae, as disclosed in U.S. Pat. No. 6,429,199 or 190 kDa protein antigen of Streptococcus mutans discussed in U.S. Pat. No. 6,541,011; M proteins or C5a peptidase of Streptococcus pyogenes disclosed in WO 2002/050107; other examples of streptococcus pyogenes include group carbohydrate antigen, C-substance, fimbrial proteins, fibronectin-binding proteins (e.g., Protein F), a cell bound streptokinase, A, B, and C streptococcal pyrogenic exotoxins, alpha C protein, beta C protein, Rib and Sip proteins, or group B carbohydrate antigens, as disclosed in U.S. Patent Publication 2006/0269541; purified capsular polysaccharide of 7 serotypes of S. pneumoniae (4,9V, 14, 19F, 23F, 18 C and 6B); pneumococcal surface protein A, pneumococcal surface adhesion A, choline binding protein A, LytB glucosaminidase, LytC muramidase, PrtA serine protease, PhtA (histidine triad A) and pneumococcal vaccine antigen A, as mentioned in WO/2004/092209; Group B streptococcal Ema (extracellular matrix adhesion protein polypeptides) EmaA, EmaB, EmaC, EmaD and EmaE from U.S. Pat. No. 7,128,919; Pac antigen of Streptococcus mutans, as mentioned in U.S. Pat. No. 6,541,011; MW antigens of Salmonella typhi, mentioned in U.S. Pat. No. 6,541,011; Treponema pallidum; Yersinia, such as V antigen or F1 antigen or pH6 antigen of Yersina pestis, disclosed in U.S. Pat. No. 6,541,011 or 37 kDa secreted polypeptide encoded on the 70 kb virulence plasmid of pathogenic Yersinia as disclosed in U.S. Pat. No. 6,541,011.

Fungal antigens Fungal pathogens may also be detected by the kit and the methods disclosed. In one example, the antigen is from Candida albicans. In one specific example, the antigen is a fungal adhesion molecule, such as a phosphomannoprotein, from Candida albicans, as mentioned in U.S. Pat. No. 6,630,146. In one example, the antigen is a fungal antigen from Absidia. In one example, the antigen is from Absidia corymbifera. In one example, the antigen is a fungal antigen from Acremonium. In one example, the antigen is a fungal antigen from Alternaria. In one example, the antigen is a fungal antigen from Aspergillus. In one example, the antigen is a fungal antigen from the species Basidiobolus. In one example, the antigen is a fungal antigen from the species Bipolaris. In one example, the antigen is a fungal antigen from the species Blastomyces. In one example, the antigen is a fungal antigen from the species Blastomyces. In one example, the antigen is a fungal antigen from Candida. One specific example of an antigen is a fungal adhesion molecule, such as a phosphomannoprotein, from Candida albicans, as mentioned in U.S. Pat. No. 6,630,146. In one example, the antigen is a fungal antigen from Candida. In one example, the antigen is a fungal antigen from Coccidioides. In one example, the antigen is from Coccidioides immitis. In one example, the antigen is a fungal antigen from Conidiobolus. In one example, the antigen is a fungal antigen from Cryptococcus. In one example, the antigen is a fungal antigen from Conidiobolus. In one example, the antigen is a fungal antigen from Cryptococcus. In one example, the antigen is from Cryptococcus neoformans. In one example, the antigen is a fungal antigen from Curvalaria. In one example, the antigen is a fungal antigen from Epidermophyton. In one example, the antigen is a fungal antigen from Exophiala. In one example, the antigen is a fungal antigen from Geotrichum. In one example, the antigen is a fungal antigen from Histoplasma. In one example, the antigen is from Histoplasma capsulatum. In one example, the antigen is a fungal antigen from Madurella. In one example, the antigen is a fungal antigen from Malassezia. In one example, the antigen is a fungal antigen from Microsporum. In one example, the antigen is a fungal antigen from Moniliella. In one example, the antigen is a fungal antigen from Mortierella. In one example, the antigen is a fungal antigen from Mucor. In one example, the antigen is a fungal antigen from Paecilomyces. In one example, the antigen is a fungal antigen from Penicillium. In one example, the antigen is a fungal antigen from Phialemonium. In one example, the antigen is a fungal antigen from Phialophora. In one example, the antigen is a fungal antigen from Prototheca. In one example, the antigen is a fungal antigen from Pseudallescheria. In one example, the antigen is a fungal antigen from Pseudomicrodochium. In one example, the antigen is a fungal antigen from Pythium. In one example, the antigen is a fungal antigen from Rhinosporidium. In one example, the antigen is a fungal antigen from Rhizopus. In one example, the antigen is a fungal antigen from Scolecobasidium. In one example, the antigen is a fungal antigen from Sporothrix. In one example, the antigen is a fungal antigen from Stemphylium. In one example, the antigen is a fungal antigen from Trichophyton. In one example, the antigen is a fungal antigen from Trichosporon. In one example, the antigen is a fungal antigen from Xylohypha.

Parasital antigens Parasital pathogens may also be detected by the kit and the methods disclosed. In one example, the antigen is a protozoan parasite and the antigen is from Babesia. In one example, the antigen is a protozoan parasite and the antigen is from Balantidium. In one example, the antigen is a protozoan parasite and the antigen is from Balantidium. In one example, the antigen is a protozoan parasite and the antigen is from Besnoitia. In one example, the antigen is a protozoan parasite and the antigen is from Cryptosporidium. In one example, the antigen is a protozoan parasite and the antigen is from Eimeria. In one example, the antigen is a protozoan parasite and the antigen is from Encephalitozoon. In one example, the antigen is a protozoan parasite and the antigen is from Entamoeba. In one example, the antigen is a protozoan parasite and the antigen is from Giardia. In one example, the antigen is a protozoan parasite and the antigen is from Hammondia. In one example, the antigen is a protozoan parasite and the antigen is from Hepatozoon. In one example, the antigen is a protozoan parasite and the antigen is from Isospora. In one example, the antigen is a protozoan parasite and the antigen is from Leishmania. In one example, the antigen is a protozoan parasite and the antigen is from Microsporidia. In one example, the antigen is a protozoan parasite and the antigen is from Neospora. In one example, the antigen is a protozoan parasite and the antigen is from Neospora. In one example, the antigen is a protozoan parasite and the antigen is from Pentatrichomonas. In one example, the antigen is a protozoan parasite and the antigen is from Plasmodium. In one example, the antigen is a protozoan parasite and the antigen is from Plasmodium. In specific examples, the antigens may include P. falciparum circumsporozoite (PfCSP), sporozoite surface protein 2 (PfSSP2), carboxyl terminus of liver state antigen 1 (PfLSA1 c-term), and exported protein 1 (PfExp-1). In one example, the antigen is from a protozoan parasite Pneumocystis. In one example, the antigen is from a protozoan parasite Sarcocystis. In one example, the antigen is from a protozoan parasite Schistosoma. In one example, the antigen is from a protozoan parasite Theileria. In one example, the antigen is from a protozoan parasite Toxoplasma. In one example, the antigen is from a protozoan parasite Trypanosoma. In other examples, the antigen is from helminth parasites. In one example, the antigen is from Acanthocheilonema. In one example, the antigen is from Aelurostrongylus. In one example, the antigen is from Ancylostoma. In one example, the antigen is from Angiostrongylus. In one example, the antigen is from Ascaris. In one example, the antigen is from Brugia. In one example, the antigen is from Bunostomum. In one example, the antigen is from Capillaria. In one example, the antigen is from Chabertia. In one example, the antigen is from Cooperia. In one example, the antigen is from Cooperia. In one example, the antigen is from Crenosoma. In one example, the antigen is from Dictyocaulus. In one example, the antigen is from Dioctophyme. In one example, the antigen is from Dipetalonema. In one example, the antigen is from Diphyllobothrium. In one example, the antigen is from Diplydium. In one example, the antigen is from Dirofilaria. In one example, the antigen is from Dracunculus. In one example, the antigen is from Enterobius. In one example, the antigen is from Filaroides. In one example, the antigen is from Haemonchus. In one example, the antigen is from Lagochilascaris. In one example, the antigen is from Loa. In one example, the antigen is from Mansonella. In one example, the antigen is from Muellerius. In one example, the antigen is from Nanophyetus. In one example, the antigen is from Necator. In one example, the antigen is from Nematodirus. In one example, the antigen is from Oesophagostomum. In one example, the antigen is from Onchocerca. In one example, the antigen is from Opisthorchis. In one example, the antigen is from Ostertagia. In one example, the antigen is from Parafilaria. In one example, the antigen is from Paragonimus. In one example, the antigen is from Parascaris. In one example, the antigen is from Physaloptera. In one example, the antigen is from Protostrongylus. In one example, the antigen is from Setaria. In one example, the antigen is from Spirocerca. In one example, the antigen is from Spirometra. In one example, the antigen is from Stephanofilaria. In one example, the antigen is from Strongyloides. In one example, the antigen is from Strongylus. In one example, the antigen is from Thelazia. In one example, the antigen is from Toxascaris. In one example, the antigen is from Toxocara. In one example, the antigen is from Trichinella. In one example, the antigen is from Trichostrongylus. In one example, the antigen is from Trichuris. In one example, the antigen is from Uncinaria. In one example, the antigen is from Wuchereria. In one example, the antigen may include the schistosome gut-associated antigens CAA (circulating anodic antigen) and CCA (circulating cathodic antigen) in Schistosoma mansoni, S. haematobium or S. japonicum. In one example, the antigen may include a multiple antigen peptide (MAP) composed of two distinct protective antigens derived from the parasite Schistosoma mansoni. In one example, the antigen may include Leishmania parasite surface molecules third-stage larval (L3) antigens of L. loa (Akue et al. (1997), Tams1-1 and Tams1-2, encoding the 30- and 32-kDa major merozoite surface antigens of Theileria annulata (Ta) and Plasmodium falciparum merozoite surface antigen 1 or 2. In one example, the antigen is Plasimodium falciparum antigen Pfs230. In one example, the antigen may include Plasimodium falciparum apical membrane antigen (AMA-I); Plasmodium falciparum proteins Pfs28 and Pfs25; Plasimodium falciparum merozoite surface protein, MSP1; the malaria antigen Pf332; Plasmodium falciparum erythrocyte membrane protein 1; Plasmodium falciparum merozoite surface antigen, PfMSP-1; Plasmodium falciparum antigens SERA, EBA-175, RAP1 and RAP2; Schistosoma japonicum paramyosin (Sj97) or fragments; and Hsp70 in parasites.

Viral antigens Viral pathogens may also be detected by the kit and the methods disclosed. In one example, the antigen is a viral antigen from an adenovirus. In one example, the antigen is a viral antigen from an alphavirus. In one example, the antigen is a viral antigen from a calicivirus. In one example, the antigen is a viral antigen from a calicivirus capsid antigen. In one example, the antigen is a viral antigen from a coronavirus. In a specific example of a coronavirus, the antigen is a SARS coronavirus. In one example, the antigen is from a cytomegalovirus. In one specific example, the antigen may include cytomegalovirus glycoprotein gB or glycoprotein gH. In one example, the antigen is a Dengue virus. In one specific example, the antigen may include a Dengue virus envelope (E) and premembrane antigens In one example, the antigen is a viral antigen from a distemper virus. In one example, the antigen is a viral antigen from an Ebola virus. In one example, the antigen is from an Epstein-Barr virus. In one specific example, the antigen is an Epstein-Barr virus (EBV) gp340 protein. In another specific example, the antigen is the Epstein-Barr virus (EBV) latent membrane protein LMP2.

In one example, the antigen is Epstein-Barr virus nuclear antigens 1 and 2. In one example, the antigen is measles virus nucleoprotein (N). In one example, the antigen is a viral antigen from an enterovirus. In one example, the antigen is a viral antigen from a flavivirus. In one example, the antigen is from Hepatitis A. In one example, the antigen is from Hepatitis B. In one example, the antigen is a viral antigen from a hepatitis B core or surface antigen. In one specific example, the antigen is Hepatitis B virus core and E antigen. In one specific example, the antigen is a hepatitis B surface antigen fused to a core antigen, core-preS2 particles. In one example, the antigen is from Hepatitis C. In one specific example, the antigen is a Hepatitis C virus nucleocapsid protein in a secreted or a nonsecreted form. In another specific example, the antigens may include the hepatitis C virus antigens: the core protein (pC); E1 (pE1) and E2 (pE2) alone or as fusion proteins. In one example, the antigen is from Herpes simplex, types I and II. In one example, the antigen is a viral antigen from a herpes simplex virus or varicella zoster virus glycoprotein. In one specific example, the antigen may include ICP0, ICP4, ICP27, ICP47, gB, gD, gE, gG, gH, and gI of the herpes simplex virus. In one example, the antigen is a viral antigen from an infectious peritonitis virus. In one example, the antigen is a viral antigen from HIV. In one specific example, the antigen may include a HIV antigen such as Gag, Po1, Vif, Nef, p24, gp120, gp 160, gp41 or gp36. In one example, the antigen is a viral antigen from an influenza virus. In one example, the antigen is from an influenza A, B or C viruses. In one specific example, the antigen is a viral antigen from an influenza A hemagglutinin, neuraminidase, or nucleoprotein. In one specific example, the antigen is N2 neuraminidase of an influenza A virus. In one example, the antigen is a viral antigen from a leukemia virus. In one example, the antigen is a viral antigen from a Marburg virus. In one example, the antigen is from a measles virus. In one example, the antigen is from the mumps virus. In one example, the antigen is a viral antigen from an orthomyxovirus. In one example, the antigen is a viral antigen from a papilloma virus. In one specific example the antigen may include the E1, E2, E3, E4, E5, E6 and E7 proteins of human papillomavirus. In one example, the antigen is a viral antigen from a parainfluenza virus. In one specific example of a viral antigen from a parainfluenza virus, the antigen is a hemagglutinin or a neuraminidase. In one example, the antigen is a viral antigen from a paramyxovirus. In one example, the antigen is a viral antigen from a pestivirus. In one example, the antigen is a viral antigen from a picorna virus. In an example of a picornavirus, the antigen may come from a coxsackievirus. In an example of a picornavirus, the antigen may come from an echovirus. In an example of a picornavirus, the antigen may come from a poliovirus. In an example of a picornavirus, the antigen may come from a rhinovirus. In one specific example of a picorna virus antigens, the antigens may include a poliovirus capsid antigen, or a pox virus antigen. In one example, the antigen is a viral antigen from a rabies virus. In one specific example, the antigens include rabies virus glycoproteins. In one example, the antigen is a viral antigen from a reovirus. In one example, the antigen is from a respiratory syncytial virus. In one specific example, the antigen is a respiratory syncytial virus fusion protein (PFP-2). In one example, the antigen is from a rubella virus. In one example, the antigen is a viral antigen from a rotavirus. In one specific example, the antigen may include rotavirus antigen VP4, VP7, or VP7sc. In another specific example, the antigen may include proteins encoded by the VP6 and VP7 genes of rotaviruses. In one example, the antigen may be from vaccinia. In one example, the antigen is from human T-lymphotropic virus. In one specific example, the antigen may include a human T-lymphotropic virus type I gag protein.

In one example, an antigen is selected to detect a non-infectious disease, such as cancer, Alzheimer's disease or other non-infectious diseases. For example, the cancer may be prostate cancer, and the antigen selected may be a prostate specific antigen (PSA). In an example of antigen from Alzheimer's disease, the antigen is an Alzheimer's disease antigen, i.e., A68, or a recombinant human tau, as described in U.S. Pat. No. 6,864,062, for example.

### EXAMPLES

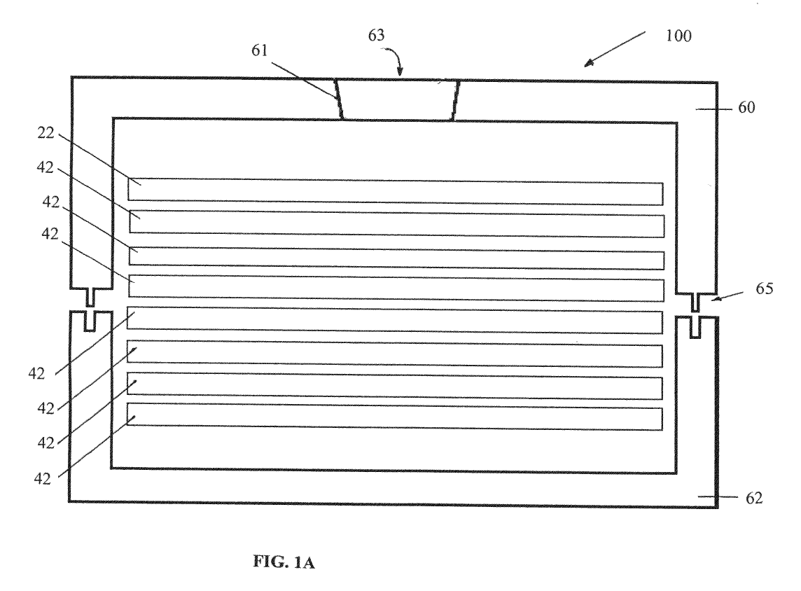

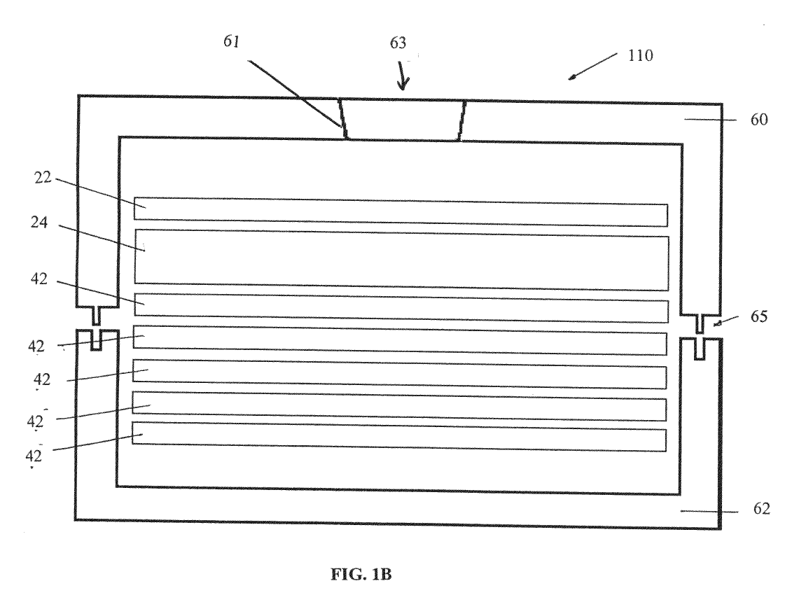

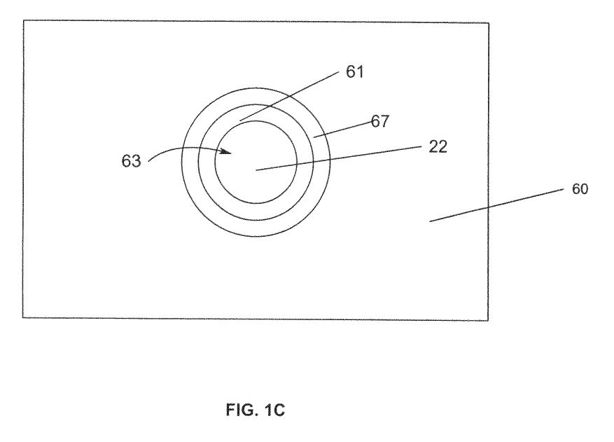

FIGS. 1A-C show a schematic example of a test kit assembly cross section. A plurality of layers 42 of an absorbent material and a membrane 22 are compressed between a cassette top 60 and a cassette bottom 62, which are represented in the drawin in an exploded view, for clarity.

As shown in FIG. 1A, a rapid test kit 100 comprises a cassette top 60 having an opening 63 and a cassette bottom 62. A wall 61 of port 63 may be angled or may be straight as shown. Additionally depicted is connection part 65, which may provide a snap or press fit, for example. A cellulose filter paper 22 may be loaded with one or more antigens. A plurality of absorbent layers 42 may be the same as the filter paper 22 or may be different. The absorbent layers 42 may have the same physical and chemical characteristics or may differ from each other, including length, absorbency and thickness. In one example of the filter paper 22 and plurality of absorbent layers 42 have a dimension of 1 inch squares. The layers may be of uneven length, width and thickness. The plurality of absorbent layers 42 may be two or more depending on their thickness and the dimensions of the cavity formed by the top 60 and the bottom 62. Preferably, the top 60 and the bottom 62 compress the layers 42 to achieve intimate physical contact one to the other. In one example, the layers 42 are of a filter paper and include five to ten layers, depending on the characteristics of the filter paper and the cassette.

For example, a cassette top 60 may be press or snap fitted onto the cassette bottom 62. A central opening or port 63, through which plasma, serum, blood, saliva or other body fluids pass through the device, includes antigens for detecting antibodies. The antigen or antigens, may be loaded before testing either before or after assembly of the kit. In FIG. 1B, a wicking pad 24 replaces one or more absorbent layers 42 of a test kit 110.

In FIG. 1C, a top plan view of a diagnostic kit is illustrated. The cassette top 60 includes an angular wall 61 defining a port 63. The length of the wall 61 may be increased by a collar 67 extending above the top 60 and providing a greater volume within the port 63.

In one example 1 μl of an antigen or antigen mixture is added at a position T (i.e., a test position) of the flow through device and 1 μl of protein A (1 mg/ml) is added at a different position C (i.e., a control position) of the test device. Then, the test device is dried. For example, 6-8 hours of air drying is sufficient for drying most test kits. A test sample, such as blood, serum or plasma, may be tested for presence of an antibody using a staining buffer. In one example, the staining buffer is Protein A coupled to colloidal gold. For example, a staining buffer may be freeze-dried for later use and may be rehydrated using a buffer solution, such as 1× Dulbecco's Phosphate Buffer Saline (DPBS), for example.

For detection of antibodies specific to a given antigen, for example, 10 μl of serum, plasma, or whole blood of a test sample may be diluted with 150 μl of dilution buffer. In one example, the dilution buffer is ACK Lysis Buffer, Cat # 1683, obtained from Invitrogen. The now diluted sample is deposited into a port 63 of a test device and onto the reaction layer 22, which may be comprised of an antigen test spot on a cellulose filter paper. For a blood sample, it is advised to wait for about three minutes after the blood is added to the dilution buffer, or at least until the dilution buffer becomes uniformly a clear red. Once the diluted sample is absorbed, 150 μl of a staining buffer may be added. In one example, the staining buffer is Protein A coupled to colloidal gold. Once the staining buffer is absorbed, 200 μl of destaining buffer may be added. The destaining buffer may be Dulbecco's Phosphate Buffer (1×) Saline (DPBS), which is also an example of phosphate buffer solution, for example. Once the destaining buffer flushes the system, results may be read immediately, without further delay, resulting in a rapid test. When both test position T and control position C appear red, a test result is positive for the presence of antibodies indicative of a particular disease, such as HIV, for example. However, if only the control position C has a red dot, then the test result is negative for the presence of antibodies associated with the disease detected by the antigen. If no dot is visible or if the control position has no dot visible, then the test is invalid. The control dot C should always be visible, if the test is properly performed.

In one example of the process, a blood sample is diluted ten fold with a lysing buffer. Samples testing positive for a specific antibody have two red dots. In another example of the process, a silver enhancing buffer is used to improve contrast.

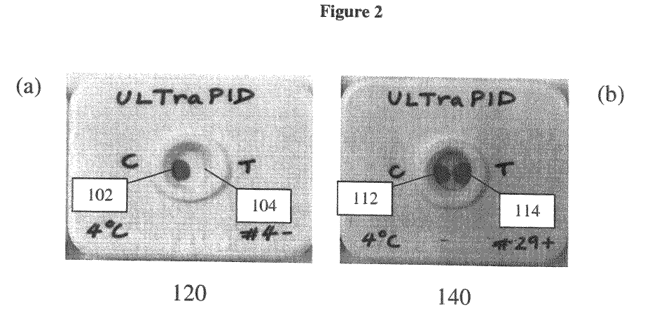

As shown in FIG. 2, for example, a first C spot 102 of a first test device 120 and second C spot 112 of a second test device 140 serve as control spots, which help to confirm that the test device is functioning properly. A first T spot 104 of the first test device 120 and a second T spot 114 of a second device 140 are test spots for detecting the presence of a specific antibody or antibodies. None of the tests performed resulted in false negatives.

In the example of FIG. 2(a), the first T spot 104 has no red spot, indicating the absence of any detectable level of antibodies in the particular test sample. In the example of FIG. 2(b) the T spot 114, shows a red spot in addition to control spot 112, positively indicating infection of the specimen with antibodies for HIV.

The following examples illustrate various types of antigens that may be used in a rapid test kit. An antibody or antibodies present in a sample may bind to the specific antigen. The examples are not intended to limit the type of antibody tested by the test kit, as any antibody that is capable of being tested in bodily fluid, such as blood, serum or plasma or other bodily fluids may be tested.

### Example 1

### Actinomyces

In one example, the antigen is Actinomyces. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 2

### Aerobacter Aerogens

In one example, the antigen is Aerobacter aerogens. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 3

### Bacillus

In one example, the antigen is Bacillus. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 4

### Bacteroides

In one example, the antigen is Bacteroides. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 5

### Bartonella

In one example, the antigen is from the species Bartonella. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 6

### Borrelia

In one example, the antigen is selected from a species of Borrelia. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 7

### Brucella

In one example, the antigen is selected from a species of Brucella. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 8

### Campylobacter

In one example, the antigen is selected from a species of Campylobacter. or detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 9

### Chlamydia

In one example, the antigen is selected from a species of Chlamydia. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 10

### Clostridium

In one example, the antigen is selected from a species of Clostridium. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 11

### Corynebacterium

In one example, the antigen is selected from a species of Corynebacterium. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μL of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 12

### H. Pylori

In one example, the antigen is selected to be H. pylori. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 13

### Helicobacter

In one example, the antigen is selected to be Heliobacter. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 14

### Hemophilus Influenzae

In one example, the antigen is selected to be Hemophilus influenzae. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 15

### Klebsiella

In one example, the antigen is selected to be from a species of Klebsiella. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 16

### Leptospira Icterohemorrhagiae

In one example, the antigen is selected to be from a species of Leptospira icterohemorrhagiae. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 17

### Leishmania Major

In one example, the antigen is selected to be from Leishmania major. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 18

### Leptospira

In one example, the antigen is selected to be from Leptospira. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 19

### Listeria

In one example, the antigen is selected to be from a species of Listeria. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 20

### Moraxella

In one example, the antigen is selected to be from a species of Moraxella. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 21

### Mycobacteria

In one example, the antigen is selected to be from a species of Mycobacteria. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 22

### Neisseria

In one example, the antigen is selected to be from a species of Neisseria. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 23

### Pasteurella

In one example, the antigen is selected to be from a species of Pasteurella. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 24

### Pneumococcus

In one example, the antigen is selected to be from a species of Pneumococcus. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 25

### Rickettsia

In one example, the antigen is selected to be from a species of Rickettsia. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 26

### Salmonella

In one example, the antigen is selected to be from a species of Salmonella. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 27

### Shigella

In one example, the antigen is selected to be from a species of Shigella. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 28

### Staphylococcus

In one example, the antigen is selected to be from a species of Staphylococcus. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 29

### Streptococcus

In one example, the antigen is selected to be from a species of Streptococcus. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 30

### Treponema Pallidum

In one example, the antigen is selected to be from Treponema pallidum. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 31

### Yersina

In one example, the antigen is selected to be from Yersina. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 32

### Candida Albicans

In one example, the antigen is selected to be from Candida albicans. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 33

### Absidia

In one example, the antigen is selected to be from Absidia. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 34

### Acremonium

In one example, the antigen is selected to be from Acremonium. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 35

### Alternaria

In one example, the antigen is selected to be from Alternaria. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 36

### Basidiobolus

In one example, the antigen is selected to be from Basidiobolus. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 37

### Blastomyces

In one example, the antigen is selected to be from Blastomyces. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 38

### Coccidioides

In one example, the antigen is selected to be from a species of Coccidioides. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 39

### Cryptococcus

In one example, the antigen is selected to be from a species of Cryptococcus. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 40

### Curvalaria

In one example, the antigen is selected to be from a species of Curvalaria. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 41

### Epidermophyton

In one example, the antigen is selected to be from a species of Epidermophyton. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 42

### Exophiala

In one example, the antigen is selected to be from a species of Exophiala. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 43

### Geotrichum

In one example, the antigen is selected to be from a species of Geotrichum. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 44

### Histoplasma Capsulatum

In one example, the antigen is selected to be from a species of Histoplasma capsulatum. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 45

### Madurella

In one example, the antigen is selected to be from a species of Madurella. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 47

### Malassezia

In one example, the antigen is selected to be from Malassezia. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 48

### Microsporum

In one example, the antigen is selected to be from Microsporum. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution; for example. Once the destaining buffer flushes the system, results may be read immediately. When both test position T and control position C appear red, a test result is positive for the presence of antibodies indicative of a particular disease, the antibodies specific for the antigen.

### Example 49

### Moniliella

In one example, the antigen is selected to be from Moniliella. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 50

### Mortierella

In one example, the antigen is selected to be from Mortierella. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 51

### Mucor

In one example, the antigen is selected to be from Mucor. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 52

### Phialemonium

In one example, the antigen is selected to be from Phialemonium. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 52

### Phialophora

In one example, the antigen is selected to be from Phialophora. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 53

### Prototheca

In one example, the antigen is selected to be from Prototheca. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 54

### Pseudallescheria

In one example, the antigen is selected to be from Pseudallescheria. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 55

### Pseudomicrodochium

In one example, the antigen is selected to be from Pseudomicrodochium. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 56

### Pythium

In one example, the antigen is selected to be from a species of Phythium. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies-specific for the antigen.

### Example 57

### Rhinosporidium

In one example, the antigen is selected to be from a species of Rhinosporidium. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 58

### Rhizopus

In one example, the antigen is selected to be from a species of Rhizopus. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 59

### Scolecobasidium

In one example, the antigen is selected to be from a species of Scolecobasidium. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 60

### Sporothrix

In one example, the antigen is selected to be from a species of Sporothrix. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 61

### Stemphylium

In one example, the antigen is selected to be from a species of Stemphylium. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 62

### Trichophyton

In one example, the antigen is selected to be from a species of Trichophyton. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 63

### Trichosporon

In one example, the antigen is selected to be from a species of Trichosporon. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 64

### Xylohypha

In one example, the antigen is selected to be from a species of Xylohypha. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 65

### Babesia

In one example, the antigen is selected to be from a species of Babesia. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 66

### Balantidium

In one example, the antigen is selected to be from a species of Balantidium. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 67

### Balantidium

In one example, the antigen is selected to be from a species of Balantidium. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 68

### Besnoitia

In one example, the antigen is selected to be from a species of Besnoitia. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 69

### Cryptosporidium

In one example, the antigen is selected to be from a species of Cryptosporidium. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 70

### Eimeria

In one example, the antigen is selected to be from a species of Eimeria. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 71

### Encephalitozoon

In one example, the antigen is selected to be from a species of Encephalitozoon. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 72

### Entamoeba

In one example, the antigen is selected to be from a species of Entamoeba.

For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 73

### Giardia

In one example, the antigen is selected to be from a species of Giardia. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 74

### Hammondia

In one example, the antigen is selected to be from a species of Hammondia. For detection of an antibody or antibodies specific to the antigen, 10 μl of serum, plasma, or whole blood of the test sample is first diluted with 150 μl of dilution-buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 75

### Hepatozoon

In one example, the antigen is selected to be from a species of Hepatozoon. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 76

### Isospora

In one example, the antigen is selected to be from a species of Isospora. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 77

### Leishmania

In one example, the antigen is selected to be from a species of Leishmania. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 78

### Microsporidia

In one example, the antigen is selected to be from a species of Microsporidia. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 79

### Neospora

In one example, the antigen is selected to be from a species of Neospora. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 80

### Pentatrichomonas

In one example, the antigen is selected to be from a species of Pentatrichomonas. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 81

### Plasmodium

In one example, the antigen is selected to be from a species of Plasmodium. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 82

### Pneumocystis

In one example, the antigen is selected to be from a species of Pneumocystis. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 83

### Sarcocystis

In one example, the antigen is selected to be from a species of Sarcocystis. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 84

### Theileria

In one example, the antigen is selected to be from a species of Theileria. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 85

### Toxoplasma

In one example, the antigen is selected to be from a species of Toxoplasma. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 86

### Trypanosoma

In one example, the antigen is selected to be from a species of Trypanosoma. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 87

### Schistosoma

In one example, the antigen is selected to be from a species of Schistosoma. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 88

### Schistosoma

In one example, the antigen is selected to be from a species of Schistosoma. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 89

### Adenovirus

In one example, the antigen is selected to be from a species of an adenovirus. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 90

### Coronavirus

In one example, the antigen is selected to be from a species of a coronavirus. The coronavirus antigen may be a SARS antigen, for example. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 91

### Cytomegalovirus

In one example, the antigen is selected to be from a species of cytomegalovirus. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 92

### Dengue Virus

In one example, the antigen is selected to be from a species of a Dengue virus. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 93

### Ebola Virus

In one example, the antigen is selected to be from a species of an Ebola virus. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 94

### Epstein-Barr Virus

In one example, the antigen is selected to be from a species of an Epstein-Barr virus. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 95

### Measles Virus

In one example, the antigen is from a species of a measle virus. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 97

### Chickenpox Virus

In one example, the antigen is from a species of a chickenpox virus. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 98

### Enterovirus

In one example, the antigen is selected to be from a species of an enterovirus. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 99

### Hepatitis A

In one example, the antigen is a Hepatitis A antigen. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 100

### Hepatitis B

In one example, the antigen is a Hepatitis B antigen. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 101

### Hepatitis C

In one example, the antigen is a Hepatitis C antigen. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 102

### Herpes Simplex

In one example, the antigen is a Herpes simplex virus. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

### Example 103

### HIV Virus

In one example, the antigen is a HIV 1 antigen such as p24 for detecting HIV-1. The p24 antigen also works for detecting a HIV-2 antigen. In this example, the p24 antigen consists of the following sequence:

PIVQNIQGQMVHQAISPRTLNAWVKVVEEKAFSPEVIPMFSALSEGATPQDLNTMLNTVGGHQAAMQMLKETIN
EEAAEWDRVHPVHAGPIAPGQMREPRGSDIAGTTSTLQEQIGWMTNNPPIPVGEIYKRWIILGLNKIVRMYSPTSI
LDIRQGPKEPFRDYVDRFYKTLRAEQASQEVKNWMTETLLVQNANPDCKTILKALGPAATLEEMMTACQGVGG
PGHKARVL

For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 104

### HIV Virus

In one example, the HIV antigen is a HIV-1 gp 41 partial protein, which consists of the following sequence:

SELYKYKVVKIEPLGVAPTKAKRRVVQREKRAVGIGALFLGFLGAAGSTMGAASMTLTV
QARQLLSGIVQQQNNLLRAIEAQQHLLQLTVWGIKQLQARILAVERYLKDQQLLGIWGCSGKLICTTAVPWNAS
WSNKSLEQIWNNMTWMEWDREINNYTSLIHSLIEESQNQQEKNEQELLELDKWASLWNWFNITNWLWYIKLFI
MIVGGLVGLRIVFAVLSVVNRVRQGYSPLSFQTHLPIPRGPDRPEGIEEEGGERDRDR

For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen. Particularly good results were obtained by using this partial protein of gp 41.

### Example 105

### HIV Virus

In one example, the antigen used to detect HIV infection is a gp41 peptide fragment which consists of the following sequence:

QLQARILAVERYLKDQQLLGIWGCSGKLICTTAVPWNAS.

For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen. Particularly good results were obtained by using this partial protein of gp 41.

Additional examples of antigen preparation may also occur before testing for the presence of an antigen. In one example of antigen preparation, an antigen preparation for a diagnostic kit comprises at least two HIV antigens, such as gp41 and p24 in a 1:1 ratio. For example, gp41 at a concentration of 1.6 mg/ml and a p24 concentration of 1.47 mg/ml may be prepared to a final concentration of 0.8 mg/ml gp41 and 0.735 mg/ml p24.

In another example of antigen preparation, an HIV-1 antigen, an HIV-2 antigen or both are used. In this example, an antigen mixture may be prepared. Peptide antigens gp41 and gp36 are dissolved in distilled H₂O at concentration of 2 mg/ml each. A p24 purified protein is suspended at a concentration of 2 mg/ml in 20 mM carbonate buffer (pH of 9.6). An antigen cocktail is prepared at ratio of gp41:gp36:p24 at a molar ratio of 5:2:3. The prepared antigen cocktail is then distributed into aliquots and kept in −20° C. degree. The antigen cocktail is immobilized on a filter paper made of a cellulose having a substantial α-cellulose content. In one example, the cellulose content is 98%, for example. A cellulose filter having a particle retention size of 20-25 μm and an ash percentage of 0.06% is used, for example. A frozen antigen cocktail may be thawed before loading to the cellulose filter paper. One microliter (μl) of antigen (about 2 μg) is loaded on the filter paper and is air-dried and stored at room temperature before assembling the antigen loaded filter paper in a test device.

In another example of antigen preparation, an antigen cocktail is prepared using peptide antigens gp41 and gp36 dissolved in distilled H₂ O at concentration of 2 mg/ml each. A p24 purified protein is suspended at a concentration of 2 mg/ml in 20 mM carbonate buffer (pH of 9.6). For example, an antigen cocktail is prepared at ratio of gp41:gp36:p24 of 5:2:3. The prepared antigen cocktail may be distributed into aliquots and kept in −20° C. degree. Frozen antigen cocktail may be thawed before loading onto a cellulose filter paper. One μl of antigen (about 2 μg) is loaded on a cellulose filter paper, which is air-dried and stored at room temperature before assembling the loaded filter paper in a test device.

In one example, two HIV-1 antigens are used. The antigens are expressed in bacterial and purified using standard molecular biology methods. They may be a HIV-1 p24 protein, as previously discussed, and a HIV-1 gp41, which may be either be the whole protein, partial protein or peptide fragment.

In one example, a homologous sequence exhibits more than 80% identity with an amino acid sequence of a gp41 peptide, for example.

### Example 106

### Influenza Virus

In one example, the antigen is an influenza viral antigen such as an influenza A, B or C antigen, for example. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies in a particular disease, the antibodies specific for the antigen.

### Example 107

### Leukemia Virus

In one example, the antigen is from a leukemia virus. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies in a particular disease, the antibodies specific for the antigen.

### Example 108

### Marburg Virus

In one example, the antigen is from a Marburg virus. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 2001 of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies in a particular disease, the antibodies specific for the antigen.

### Example 109

### Mumps Virus

In one example, the antigen is a Mumps viral antigen. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies in a particular disease, the antibodies specific for the antigen.

### Example 110

### Papilloma Virus

In one example, the antigen is a papilloma virus. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies in a particular disease, the antibodies specific for the antigen.

### Example 111

### Paramyxovirus

In one example, the antigen is a species of paramyxovirus. For detection of an antibody or antibodies specific to the antigen, 10 μl of serum, plasma, or whole blood of the test sample is first diluted with 150 μl of dilution buffer. The 150 μL of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies in a particular disease, the antibodies specific for the antigen.

### Example 112

### Pestivirus

In one example, the antigen is a species of pestivirus. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 2001 of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies in a particular disease, the antibodies specific for the antigen.

### Example 113

### Picorna

In one example, the antigen is a picorna viral antigen. In one specific example of a picorna virus antigens, the antigens may include a poliovirus capsid antigen, or a pox virus antigen. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies in a particular disease, the antibodies specific for the antigen.

### Example 114

### Rabies Virus

In one example, the antigen is a rabies viral antigen. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies in a particular disease, the antibodies specific for the antigen.

### Example 115

### Reovirus

In one example, the antigen is a reovirus antigen. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies in a particular disease, the antibodies specific for the antigen.

### Example 116

### Respiratory Syncytial Virus

In one example, the antigen is a respiratory syncytial viral antigen. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies in a particular disease, the antibodies specific for the antigen.

### Example 117

### Rubella

In one example, the antigen is a rubella viral antigen. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies in a particular disease, the antibodies specific for the antigen.

### Example 118

### Rotavirus

In one example, the antigen is a rotavirus antigen. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies in a particular disease, the antibodies specific for the antigen.

### Example 119

### Vaccinia

In one example, the antigen is a vaccinia viral antigen. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies in a particular disease, the antibodies specific for the antigen.

### Example 120

### Human T-Lymphotropic Virus

In one example, the antigen is a human T-lymphotropic viral antigen. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies in a particular disease, the antibodies specific for the antigen.

### Example 121

### Prostate Cancer

In one example, the antigen is from a non-infectious disease, such as cancer. In a specific example of a cancer, the cancer is prostate cancer and the antigen is a prostate specific antigen (PSA). For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies in a particular disease, the antibodies specific for the antigen.

### Example 122

### Alzheimer's Disease

In one example, the antigen is an A68 antigen, from Alzheimer's disease. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies in a particular disease, the antibodies specific for the antigen.

### Example 123

### Combination of Viral and Bacterial Antigens

In one example, two or more antigens may be detected by the test kit. The two different antigens may be a viral and a bacterial antigen, for example. The bacterial antigen may be a Mycobacterium Tubercolis. The viral antigen may be a Hepatitis antigen or a HIV antigen, for example. For detection of each antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies in a particular disease, the antibodies specific for the antigen.

Combinations that are tested using the test kit are not merely a combination of viral and bacterial antigens. In another example, a combination of two or more viral antigens may be tested. In another example, a combination of viral, parasital, bacterial and fungal antigens may be selected. In one example of type of combination of viral, parasital, bacterial and fungal antigen, a combination of fungal and viral infections may be tested. The combinations described herein are not limited to the specific examples disclosed.

In one example, a plurality of two or more test dots will be present, indicating that the test result is positive for the presence of antibodies for two or more particular diseases. Thus, two test dots testing for a combination of viral and bacterial antigens indicate that the person has antibodies for a viral disease and a bacterial disease.

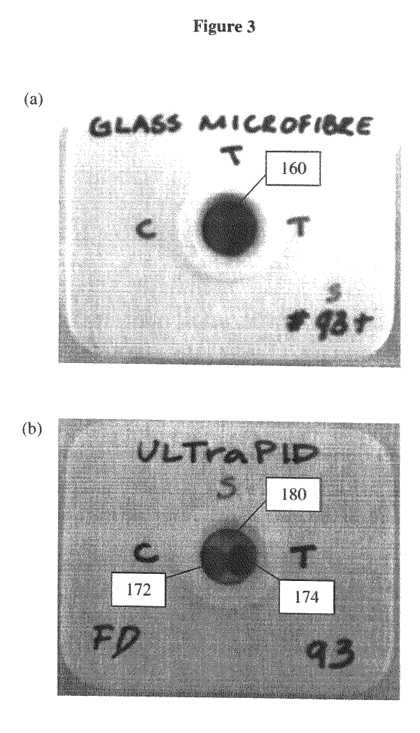

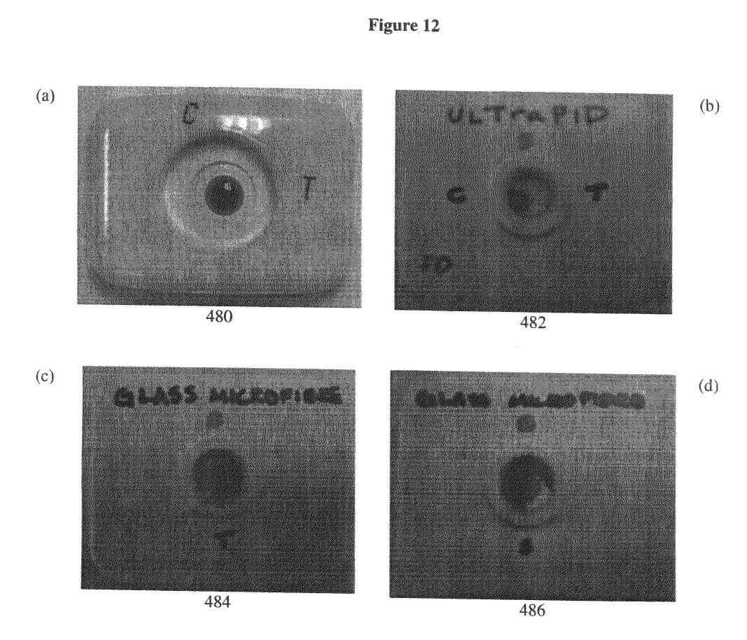

In FIG. 3 (a) and (b), results of a sample using a glass fiber membrane 160 are compared to a rapid test kit using one or more HIV antigens for detecting the presence of HIV in a sample of plasma. The glass fiber membrane 160 failed because plasma could not flow through. In comparison, a sample using a cellulose filter paper 180, otherwise similar in physical characteristics, clearly indicates a test positive for HIV with much better contrast. The control spot 172 is apparent, and positive test spot 174 matches the color index value of the control spot 172. The characteristics of glass fiber membranes utilized for this test are described in Table 9, which follow the description of FIG. 12.

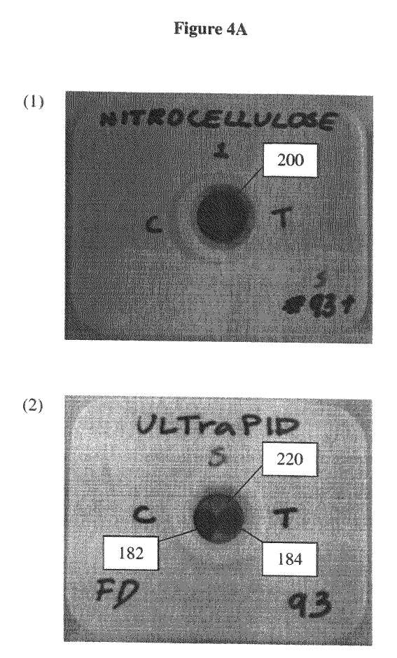

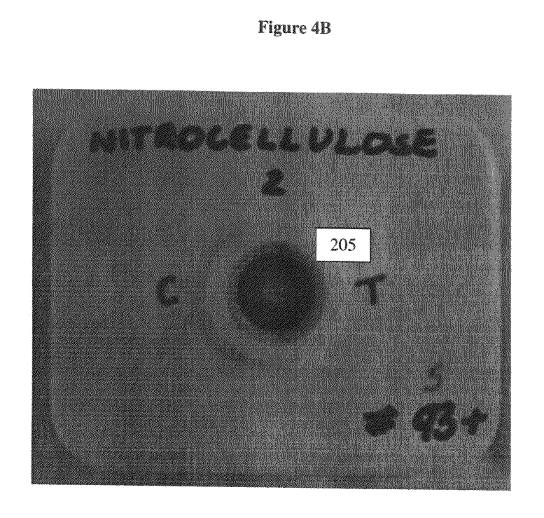

In FIG. 4A (1), a plasma sample tested using a nitrocellulose membrane 200 is compared with a sample tested using a cellulose filter paper 220. The nitrocellulose membrane as shown in FIG. 4A(1) fails, providing poor contrast and requiring a longer time to perform the test than for the rapid test kit using cellulose reaction layer 220. The red residue is the colloidal gold solution that did not pass through the reaction membrane. The nitrocellulose membrane, obtained from Bio-Rad Laboratories, had a pore size of 0.45 microns using a cellulose reaction layer 22, as shown in FIG. 4B(2), the control spot 182 is clearly evident, as is a positive test spot 184, which matches the color index value of the control spot.

In FIG. 4B, a device 205 uses a nitrocellulose mixed ester membrane having a pore size of 5 microns. Plasma fails to flow through, causing this membrane to fail also.

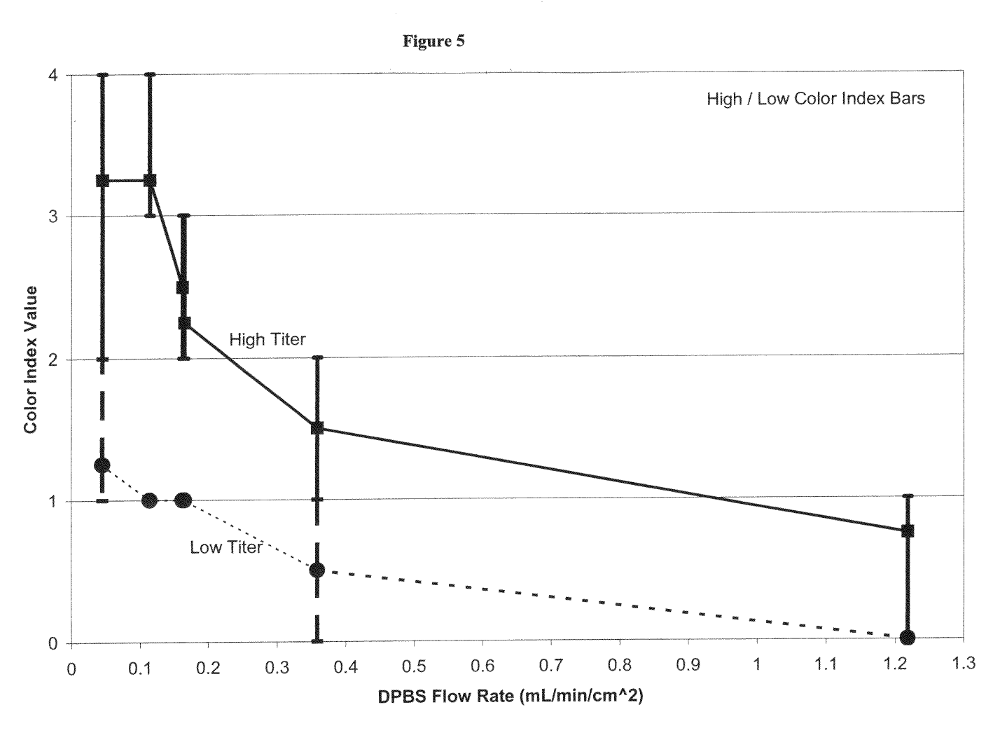

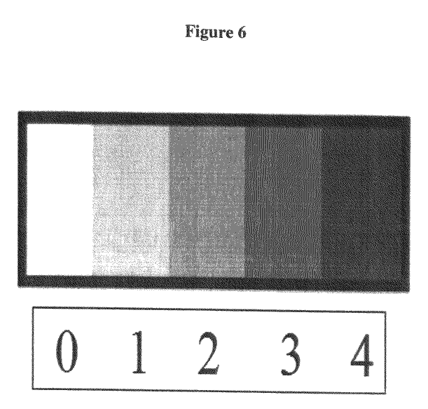

In FIG. 5, a flow rate of PBS is measured using a modified ASTM Standard for measuring flow rate through a 7 cm circle of filter paper folded in quarters and suspended in a ring. Then, several filter papers were used to make test kits using the same antigens and loading. Tests were performed using HIV positive samples, and the color intensity of test spots were determined using the color index value chart of FIG. 6. FIG. 5 shows a graph of color index value versus DPBS flow rate. Measurements are shown for water and phosphate buffered saline, using qualitative filter paper and wet strengthened filter paper having various ratings for particle retention size. Data for FIG. 5 is found in Table 2. The error bars in FIG. 5 represent high and low data values. High titer samples are shown with squares and low titer with circles.

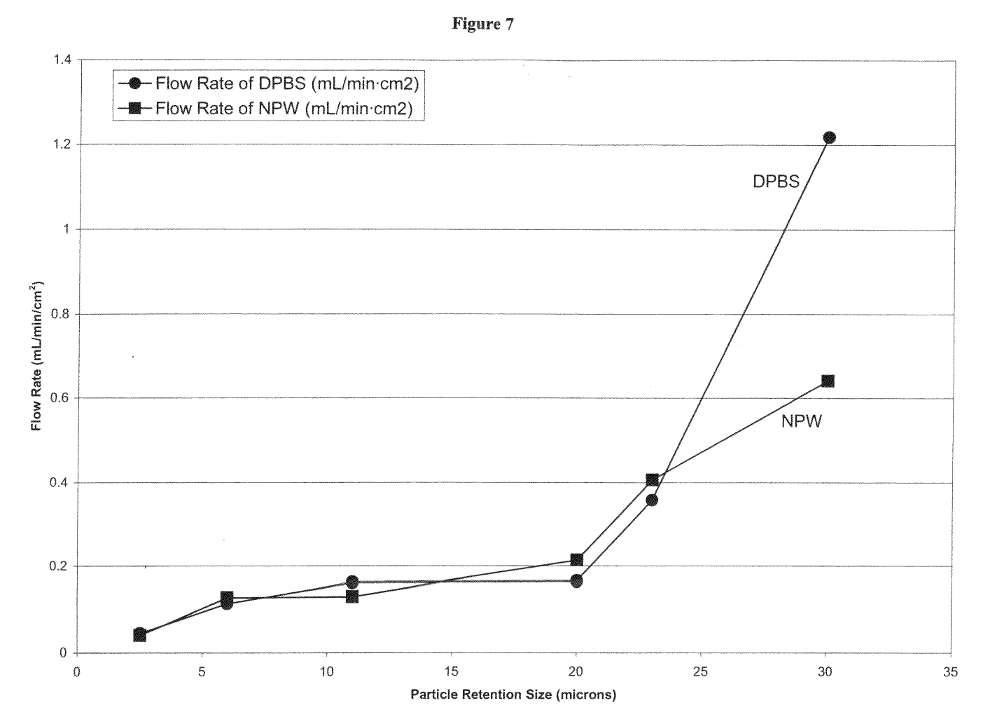

Flow rate is correlated with color index values. The low flow rates are more sensitive than higher flow rate membranes. In this example, six different types of Whatman™ filter paper were tested; each having particle retention sizes ranging from 2.5 microns to 30 microns. Surprisingly, as shown in FIG. 7, the flow rate showed a plateau region between about 6 to 20 microns with a flow rate of about 0.1 to 0.2 mL/min/cm². The plateau region corresponded to an optimal combination of sensitivity and flow rate for testing samples, whether based on blood, serum or plasma, in one example of an HIV antibody sensitive test kit.

A modified ASTM Standard measurement was used to determine the flow rate of each membrane. Filter paper was dimensioned to a 7 cm diameter circle. The paper was placed in filtering solution (both PBS and water were tested) for a time sufficient, for the paper to be completely soaked. Then the paper was placed flat in a funnel, except for edges, which were folded upwards. Then, 5 ml of filtering solution was added to the center of the funnel and time was measured using a stopwatch. When an amount of the filtering solution had passed through the filter, the time was recorded.

The flow rate, in units of mL/min/cm² is calculated in the following manner: V/T×60s/1 min×1/cm² where V=volume in ml, T=time in seconds, s=seconds, min=minute, and surface area expressed as cm².

Table 1A shows measurements of flow rate for a given particle retention size:

| | Flow Rate of DPBS | Flow Rate of Water |
|---|---|---|
| Particle Retention Size (μm) | (mL/min/cm²) | (mL/min/cm²) |
| 2.5 | 0.045 | 0.040 |
| 6 | 0.114 | 0.128 |
| 11 | 0.162 | 0.130 |
| 20-25 | 0.165 | 0.214 |
| 23 | 0.358 | 0.406 |
| 30 | 1.218 | 0.641 |

A filter paper of qualitative type had pore sizes of 2.5, 6, 11 and 20-25 microns (which we have graphed as 20 microns). A wet strengthened filter paper had a particle retention size of 23 and 30 microns. The flow rates in water for a filter paper with a particle retention range from 2.5 microns to 23 microns is in the range of about 0.04 mL/min/cm² to about 0.4 mL/min/cm². The flow rates in DPBS are also in the range of about 0.04 mL/min/cm² to 0.4 mL/min/cm². It is not clear that there is any statistical significances in the measured differences between water and PBS. The term “about”, as it is used with flow rates, takes into consideration experimental errors introduced in any measurement as is known to a person of ordinary skill in the art.

The flow rates for nitrocellulose mixed ester membranes are much higher than those measured for cellulose filter paper, as measured in units of mL/min/cm² and shown in Table 1B, below.

For example, the contrast between data measured in Table 1A and reported in Table 1B are striking when comparing flow rates in water for a wet strengthened cellulose filter paper of 23 microns with a nitrocellulose mixed ester membrane with a pore size of 5 microns. The nitrocellulose mixed ester membrane had a flow rate several orders of magnitude greater. Previously, as shown in FIG. 4B, a sample using a nitrocellulose mixed ester membrane having a pore size of 5 microns failed. In addition, the flow rates for a paper-backed nitrocellulose having a pore size of 0.45 microns resulted in a much faster flow rate of 6 ml/min/cm², as reported by Chan et al., in paragraph [0171] of U.S. Patent Publication No. 2004/0002063.

Table 1B shows flow rate data for nitrocellulose mixed ester membranes having various pore sizes.

Typical Characteristics

Flowrates

| Pore Size | Water | Air | Bubble Point |
|---|---|---|---|
| 0.10 μm | 6.5 | 0.8 | 100 psi |
| 0.22 μm | 18.5 | 2 | 50 psi |
| 0.45 μm | 50 | 4 | 30 psi |
| 0.65 μm | 125 | 9 | 17 psi |
| 0.80 μm | 195 | 17 | 15 psi |
| 1.2 μm | 290 | 20 | 12 psi |
| 5.0 μm | 550 | 34 | 7 psi |

The nitrocellulose mixed ester membrane filters used were Magna™ Nitrocellulose mixed ester membrane filters, manufactured by GE Infrastructure Water and Process Technology. The pore size used in the example is 5 microns. The flow rate of nitrocellulose mixed ester membrane in water was measured in mL/min/cm² measured at 520 mmHg (10 psi), at 20 degrees Celsius. The air flow rate is measured in units of L/min/cm² of filtration area, measured at 520 mmHg (10 psi), at 20 degrees Celsius (68 degrees Fahrenheit). The Bubble Point pressure occurs at which air is first forced through pores of water-wet membrane.

Properties of cellulose filter papers is shown in Table 1C. Table 1C, obtained from a Whatman web site shows typical properties of cellulose filters tested, such as particle retention liquid, and airflow rate. Such properties may be used to select for a particular filter paper.

Grades 1, 3, 4, 5, 113 and 114, as reported in Table 1C below, were utilized in preparing test kits. Wet strengthened qualitative cellulose filters contain a small quantity of a chemically stable resin to give improved wet strength. For these tests, filter paper is cut down into circles with a diameter of 7 cm for flow rate measurements, and the filter papers were dimensioned to 1 inch by 1 inch squares for use in test kits.

TABLE 1C
Typical Properties of Cellulose Filters

| | | | | | | | | |
|---|---|---|---|---|---|---|---|---|
| | Particle | | | Typical | Basis | | | Tensile |
| | Retention* | Air Flow Rate | | Thickness | Weight | Wet Burst | Dry Burst | M/D Dry |
| Grade | Liquid (μm) | (s/100 mL/in²) | Ash (%) | (μm) | (g/m²) | (psi) | (psi) | (N/15 mm) |

Qualitative

| | | | | | | | | |
|---|---|---|---|---|---|---|---|---|
| 1 | 11 | 10.5 | 0.06 | 180 | 88 | 0.3 | 16 | 39.1 |
| 2 | 8 | 21 | 0.06 | 190 | 103 | 0.7 | 16 | 44.6 |
| 3 | 6 | 26 | 0.06 | 390 | 187 | 0.5 | 28 | 72 |
| 4 | 20-25 | 3.7 | 0.06 | 205 | 96 | 0.7 | 10 | 28.4 |
| 5 | 2.5 | 94 | 0.06 | 200 | 98 | 0.4 | 21 | 55.6 |
| 6 | 3 | 35 | 0.2 | 180 | 105 | 0.3 | 15 | 39.1 |

General Purpose and Wet Strengthened Qualitative

| | | | | | | | | |
|---|---|---|---|---|---|---|---|---|
| 91 | 10 | 6.2 | N/A | 205 | 71 | 2 | 18 | 28 |
| 93 | 10 | 7 | N/A | 145 | 67 | 2.6 | 12 | 38 |
| 113 | 30 | 1.3 | N/A | 420 | 131 | 8 | 24 | 38.6 |
| 114 | 23 | 5.3 | N/A | 190 | 77 | 8.9 | 15 | 42.1 |

Table 2 shows the respective color index values for each low titer and high titer sample tested at a respective particle retention size and flow rate. FIG. 5 shows mean color index and the color index bars show high and low values of the color index. Samples having a color index value of 1 are considered to be low titer samples, while samples having a color index value greater than 2 considered to be high titer samples. The cellulose filter paper with about a 1.2 mL/min/cm² flow rate failed on each low titer sample. At a flow rate of about 0.4 mL/min/cm², a rapid test kit successfully detected a high titer, HIV-positive sample, but the same test kit failed two times in four tests to detect the presence of a low titer, HIV-positive sample. Thus, for cellulose reaction layers, of flow rate of about 0.4 mL/min/cm² is an upper limit for application as an HIV screening test. Surprisingly, this rapid test kit also exhibited sufficient sensitivity and specificity to be used to distinguish between low and high titer HIV-positive samples. The data in FIG. 5 show that the color index values roughly fall off exponentially with the flow rate of cellulose filter paper. Thus, it is believed that even higher flow rates would result in more failures.

Table 2 describes the data for low and high titer samples. DPBS flow rates, particle retention sizes and results for test and control samples are shown.

TABLE 2

| DPBS Flow | Particle | | | | | | | | |
|---|---|---|---|---|---|---|---|---|---|
| Rate | Retention | Test | | | | Control | | | |
| (mL/min./cm²) | Size (μm) | 1 | 2 | 3 | 4 | 1 | 2 | 3 | 4 |
| Low Titer | | | | | | | | | |
| 0.045 | 2.5 | 1 | 2 | 1 | 1 | 4 | 4 | 3 | 2 |
| 0.114 | 6 | 1 | 1 | 1 | 1 | 3 | 4 | 3 | 3 |
| 0.162 | 11 | 1 | 1 | 1 | 1 | 3 | 3 | 2 | 2 |
| 0.165 | 20-25 | 1 | 1 | 1 | 1 | 2 | 3 | 2 | 2 |
| 0.358 | 23 | 0 | 0 | 1 | 1 | 1 | 1 | 2 | 2 |
| 1.218 | 30 | 0 | 0 | 0 | 0 | 1 | 1 | 0 | 1 |
| High Titer | | | | | | | | | |
| 0.045 | 2.5 | 4 | 4 | 3 | 2 | 4 | 4 | 4 | 4 |
| 0.114 | 6 | 3 | 4 | 3 | 3 | 4 | 4 | 4 | 4 |
| 0.162 | 11 | 3 | 3 | 2 | 2 | 4 | 4 | 4 | 4 |
| 0.165 | 20-25 | 2 | 3 | 2 | 2 | 4 | 3 | 4 | 4 |
| 0.358 | 23 | 1 | 1 | 2 | 2 | 4 | 4 | 4 | 4 |
| 1.218 | 30 | 1 | 1 | 0 | 1 | 1 | 1 | 1 | 1 |
| HIV Negative | | | | | | | | | |
| 0.114 | 6 | 0 | 0 | 0 | 0 | 4 | 4 | 4 | 4 |

FIG. 6 illustrates a color index for semi-quantitative determination of the sensitivity by measuring color index values. Anything darker than background is a 1 and so forth based on the color index of a spot. A value of 1 or greater is deemed a positive test result. A color index value greater than 2 corresponds to the high titer samples.

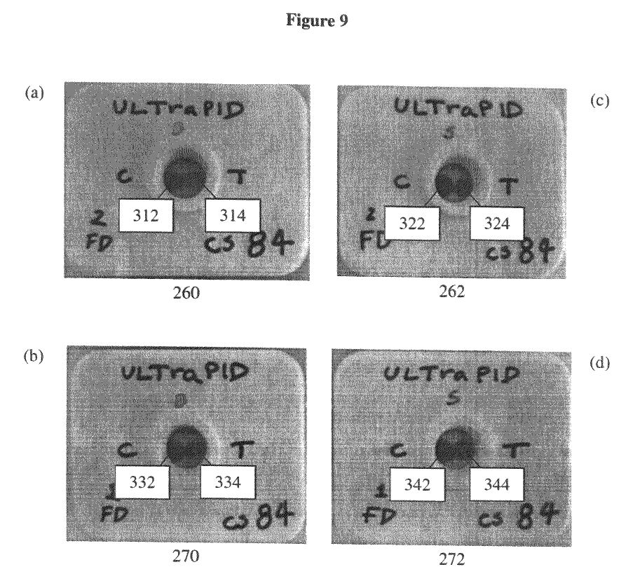

In FIG. 6, which is an example of a color index chart, the scale runs from 0, which is negative for the presence of an antibody or antibodies specific for a given antigen, to a 4, which is the highest semi-quantitative value. An index value of 0 indicates that pink staining of the background may occur but does not indicate presence of a discernable dot. An index value of 1 is distinguishable from the background, but is not darker than the color represented by 1. An index value of 2 indicates a clearly visible dot darker than 1, but not darker than 2. A value of 3 is a highly intense dot darker than 2 but not darker than the reference provided at 3. A color index value of 4 is darker than the reference labelled 3. For example, comparison of plasma and blood samples obtained from the same donor sample are shown in FIG. 9, for example. Blood tests (a), (b), have control spots 312, 332 and test spots 314, 334 comparable in color index value to the control spots 322, 342 and test spots 324, 344 of plasma samples (c), (d). Thus, both whole blood and plasma may use the same test kit with the same color index value chart.

Unless specified otherwise, comparisons with other test kits are made between commercial kits and examples using a cellulose filter paper having a flow rate of about 0.1 mL/min/cm².

In FIG. 7, a graph of flow rate versus particle retention size is presented for the data shown in Table 2 previously. Increased pore size shows increased flow rate, but increased flow rate, has decreased assay sensitivity, as shown in FIG. 5. Preferably, the sensitivity yields results capable of distinguishing high and low titer, while also providing as rapid a test as possible.

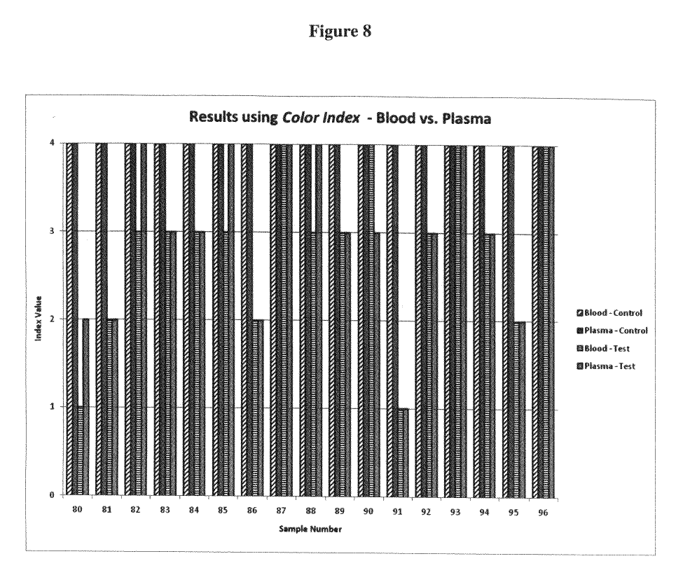

FIG. 8 shows a comparison of tests using samples of blood and plasma. The color index values are measured. The results for blood and plasma tests are remarkably similar which is very surprising and unexpected. Most tests kits cannot be used to test whole blood. As can be seen with other types of reaction membranes tested in the figures, all of the others are inoperative when used with the blood rather than plasma or serum. Use of whole blood allows testing to be conducted in the field where centrifuges are not easily available, and represents a substantial advantage over other test kits.

Table 3 shows data reported graphically in FIG. 8. Some samples were tested twice, while other samples were tested once. Table 3 compares data for samples using blood and samples using plasma, from the same source and using the same type of cellulose reaction layer.

TABLE 3

| | BLOOD | | PLASMA | |
|---|---|---|---|---|
| Sample | Trial 1 | Trial 2 | Trial 1 | Trial 2 |

| Number | C | T | C | T | C | T | C | T |
|---|---|---|---|---|---|---|---|---|
| 80 | 4 | 1 | 4 | 1 | 4 | 2 | 4 | 2 |
| 81 | 4 | 2 | 4 | 1 | 4 | 2 | 4 | 2 |
| 82 | 4 | 3 | 4 | 4 | 4 | 4 | 4 | 4 |
| 83 | 4 | 3 | 4 | 3 | 4 | 3 | 4 | 3 |
| 84 | 4 | 3 | 4 | 3 | 4 | 3 | 4 | 3 |
| 85 | 4 | 3 | 4 | 3 | 4 | 4 | 4 | 4 |
| 86 | 4 | 2 | — | — | 4 | 2 | — | — |
| 87 | 4 | 4 | — | — | 4 | 4 | — | — |
| 88 | 4 | 3 | — | — | 4 | 4 | — | — |
| 89 | 4 | 3 | — | — | 4 | 3 | — | — |
| 90 | 4 | 4 | — | — | 4 | 3 | — | — |
| 91 | 4 | 1 | — | — | 4 | 1 | — | — |
| 92 | 4 | 3 | — | — | 4 | 3 | — | — |
| 93 | 4 | 4 | — | — | 4 | 4 | — | — |
| 94 | 4 | 3 | — | — | 4 | 3 | — | — |
| 95 | 4 | 2 | — | — | 4 | 2 | — | — |
| 96 | 4 | 4 | — | — | 4 | 4 | — | — |

Plasma, serum and blood samples all have similar visual results. For example, a comparison of plasma and blood samples is shown in FIG. 9. Examples using (a), (b) blood and (c), (d) plasma are accordingly shown. These photos represent test sample 84, as reported in the tabulated data of Table 3. Test samples 260 and 270 used blood. The test kits 260 and 270 tested positive for presence of HIV, indicated by respective test spots 314, 334 and control spots 312 and 332. Test samples 262 and 272 used plasma. The test samples 262 and 272 tested positive for presence of HIV, indicated by respective test spots 324 and 344 and control spots 322 and 342.

In addition, test samples 260 and 262 were obtained from the same donor sample. One used blood while the other used plasma. Similarly, test samples 270 and 272 were obtained from the same donor sample, with one for blood and one for plasma. Both blood and plasma samples tested 3 on the color index scale.

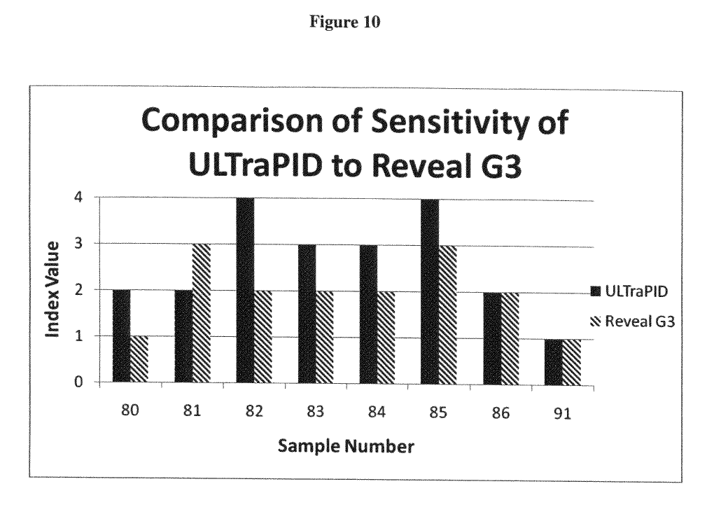

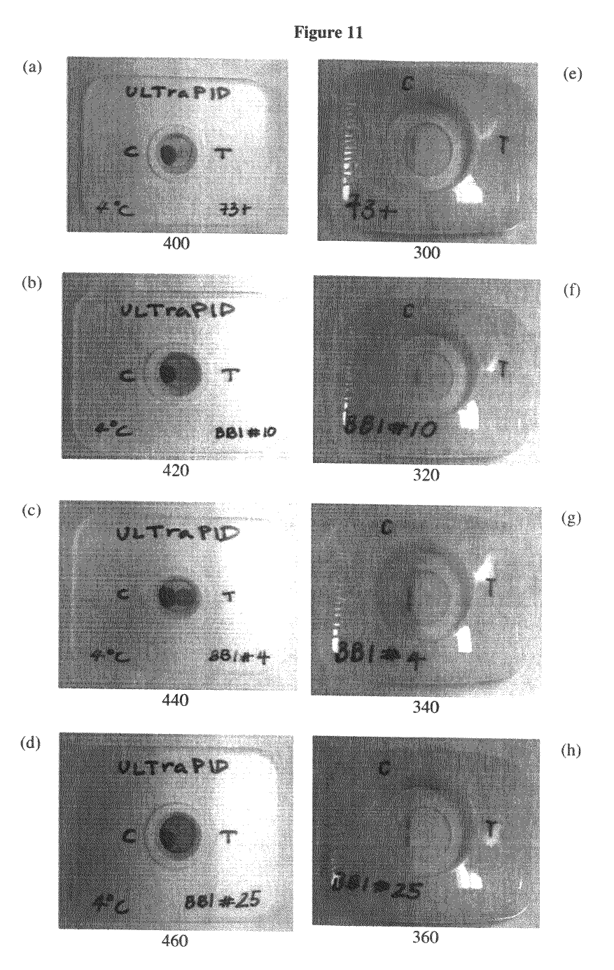

FIG. 10 shows a bar graph representing color index values for various samples using a rapid test kit with a flow rate of about 0.1 mL/min/cm² in DPBS and a commercial assay, using the Reveal® G3. Most of the plasma samples using the test kit had better contrast than plasma samples using MedMira® Reveal® G3 test kit.² In FIG. 11, some representative comparisons of a rapid test kit with a Reveal® G3 kit are shown. Rapid test kits having cellulose filter paper with a flow rate of about 0.1 mL/min/cm² in DPBS were tested. The procedure for using a rapid test kit includes adding 150 microliters of Phosphate Buffer Saline (PBS) solution is added to a freeze dried staining buffer. 10 microliters of plasma are diluted with 150 microliters of PBS solution. The kit is loaded with the diluted plasma, 150 microliters of staining buffer, and 200 microliters of PBS solution in succession. The test duration is less than two minutes, qualifying as a rapid test. For a rapid test kit, blood and serum, alternatively, may be used, in addition to plasma. This is not the case for other commercial test kits. ² MedMira® and Reveal® are registered trademarks of MedMira Laboratories, Inc., Toronto, Canada.

The Reveal® G3 kit used 3 drops of Universal Buffer added to the kit, followed by 1 drop of plasma. Then 3 drops of Universal Buffer are added to the kit. Then an instant gold cap was added on the kit, with 12 drops of Universal Buffer added through the cap. Optionally, an additional 3 drops of Universal Buffer may be added. The test duration is less than three minutes. The term “Universal Buffer” is used in the instructions for the Reveal® G3 kit. Test kit 400 tested HIV positive, which is the same result as the test kit 300 of Reveal® G3. Both kits tested 1 on the color index scale. The Reveal® G3 of FIG. 11(f) shows a G3 test kit 320 that tested patient sample BBI #10 as negative. Rapid test kit 420 for sample BBI #10 tested positive, having a color index of 1. Thus, the rapid test kit 420 indicated HIV-positive even though the Western blot showed indeterminate. Like BBI # 4 from test kit 440, and BBI #25 from sample 460, sample BBI #10, was from an Anti-HIV-1 PRB204 performance panel purchased from BBI Diagnostics, which had tested the panel on different kits. A comparison of BBI #10 with other competing kits showed that sample BBI #10 is positive using an Abbott Determine™ HIV-1/2. Other kits such as OraQuick® and Uni-Gold™ tested negative.³ A Western blot test of BBI #10 was indeterminate. BBI refers to screening assay PRB 204, which is shown in Tables 4 and 5. While Western blot is the gold standard, an indeterminate Western blot fails to identify either a positive or negative test result for HIV. 3 OraQuick® is a registered trademark of Orasure Technologies; Uni-Gold™ is a trademark of Trinity Biotech.

Test kit 440 containing sample BBI #4 tested HIV positive like test kit 340 using Reveal® G3. Rapid kit 440 tested 3 on the color index scale using cellulose reaction layer, while Reveal® kit 340 tested 1 on the same color index scale, showing better contrast for the cellulose test kit 440, making the test kit 440 easier to read. Other kits such as OraQuick® and Uni-Gold® also tested positive. A Western blot panel data also resulted in a positive result. Accordingly, for positive test results, the rapid test kit example using cellulose filter paper correctly identified a test result positive for HIV

A Reveal® G3 kit 360 using sample BBI #25 tested negative. On the color index scale used by us, the kit 360 tested 1 on a color index scale. Test kit 460 tested negative and 0 on a color index scale. A third kit, one from Abbott, for the same sample BBI #25 showed a positive result. Both OraQuick® and Uni-Gold™ tested positive. The Western blot test was indeterminate. However, the test kit, unlike Reveal® G3, allows use of blood in addition to serum or plasma. Serum and plasma samples, require laboratory equipment and more time to prepare the samples than blood. The membrane used in the test kit also absorbs quickly. From a color index measurement, this test determined that the test kit sample 460 was low titer with a color index value of 1. Thus, a rapid test kit may be used for screening, and using a color index scale, may also serve as a qualitative assay of antibody titer.

Further results for all the BBI samples tested, and a comparison of the test kit with Western blot results and competing test kits are shown in Tables 4-6. For example, BBI #1 corresponds to PRB 204-01, BBI #2 corresponds to PRB 204-02, and so forth. Tables 7 and 8 report the band patterns for the Western blot tests from the BBI panel. The Western blot band patterns are shown in Table 5 for each of the samples in the assay.

In FIG. 12, the Reveal® G3 (a) kit having a nitrocellulose membrane is shown with a sample of blood. The test with blood using the Reveal® G3 kit (a) 480 failed, while a rapid test kit (b) successfully found the sample to be negative for HIV. Blood did not flow through, but instead coagulates. This rapid test kit contained filter paper with a flow rate of about 0.1 mL/min/cm² in DPBS.

In addition, a glass fiber membrane was tested. Glass fiber membranes 484, 486 are shown in FIG. 12 (c) and (d). Blood did not flow through the membrane but instead coagulated on the surface. The glass fiber membrane that is used is a Whatman® GF/C. While Chan, in U.S. Patent Publication No. 2004/0002063, taught the use of glass fiber membranes for use with blood samples, FIG. 12 (c) and (d) clearly show that using a glass fiber membrane in this test kit failed while testing whole blood, without using the complex procedure of Chen.

The Reveal® G3 test kit also cannot utilize blood samples, completely failing in that regard. Like the Reveal® G3 test kit, the test kit of Mahajan in U.S. Patent Publication No. 2004/0023210, utilizes a nitrocellulose membrane, and also limits its use to serum and plasma. Thus, a rapid test kit is capable of better contrast using any source of antibodies including blood, plasma and serum, which is a significant and important improvement for a rapid test kit.

Nitrocellulose is well-known in the art for binding proteins, which is why it is routinely used in Western blots and other assays. However, none of these nitrocellulose assays use cellulose reaction membranes, and none are suitable as a rapid assay for use with whole blood. Alternatives to nitrocellulose are seldom considered for use in test kits. In the tests conducted with blood it is clear that nitrocellulose fails, while cellulose selected in an operative flow rate range, such as 0.04-0.4 mL/min/cm² works as well with blood as with plasma and serum. The added flexibility makes the test suitable for use as a field test. Surprisingly, there is no loss of sensitivity or specificity with the use of blood in some example test kits used for testing HIV-positive samples.

Table 6 shows characteristics of a glass fiber membrane and shows data for glass fiber membranes. For particle retention, the following is assumed: 2% initial penetration values using solid particulates dispersed in water. (Represents complete retention in normal laboratory analysis.). For flow rate, the following is assumed: Vacuum filtration of prefiltered water through 2 1/16 in. (5.5 cm) flat filter at 100 mmHg (1.9 psi). Water absorption assumes that there is an equilibrium volume of water absorbed by filter.

TABLE 6

| | |
|---|---|
| Whatman Grade | GF/C |
| Particle Retention | 1.2 μm |
| Flowrate | 10.5 sec./100 mL |
| Thickness | 0.26 mm |
| Weight | 53 g/m² |
| Max. Temp. | 500° C. (932° F.) |
| Water Absorption | 250 mL/m² |
| Sterilization | Autoclavable |

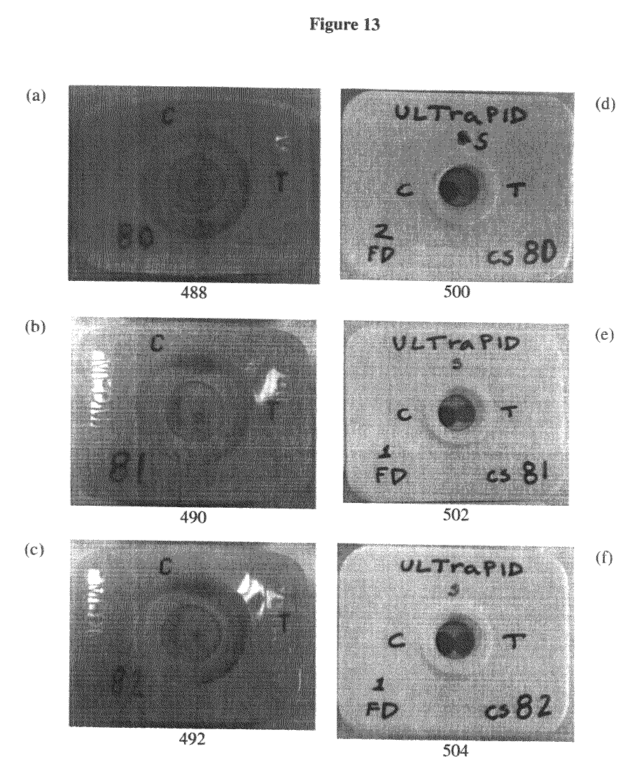

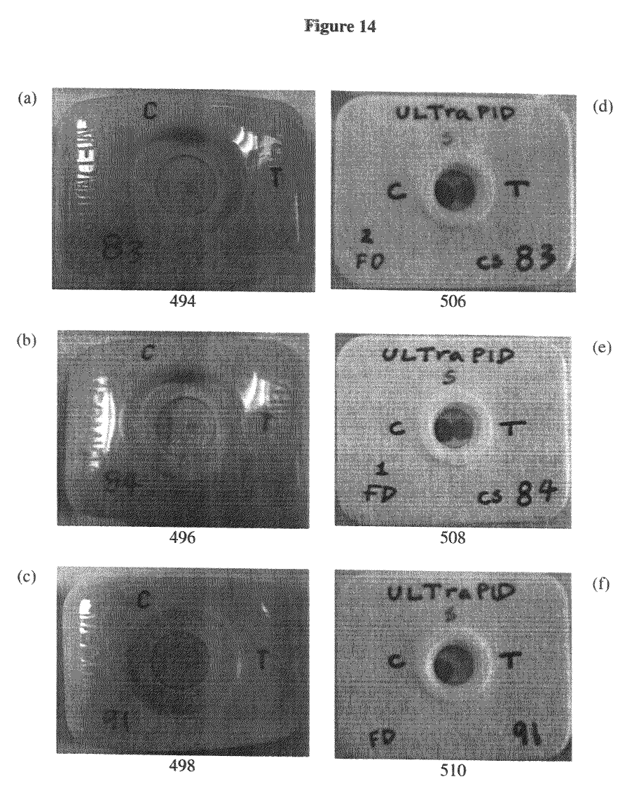

In FIGS. 13(a)-(f) and 14(a)-(f), example rapid kits are compared to Reveal® G3 kits using high and low titer samples of blood plasma. All test kits shown in the examples us a cellulose filter paper selected with a PBS flow rate of about 0.1 mL/min/cm², unless otherwise specified herein. Example test kit 500, 502, 504, 506, 508, 510 used specimens #80, #81, #82, #83, #84, and #91, respectively, and had better visual contrast than corresponding Reveal® kits 488, 490, 492, 494, 496 and 498. Color index values for samples of FIGS. 13 and 14 and some additional tests, using the color index scale of FIG. 6 are shown in Table 7. The data show that all the test kits, except for one, sample #81, had better visual contrast than Reveal® G3 kits for the same plasma samples tested.

TABLE 7

| Sample Number | Test Kit | MedMira Reveal ® G3 |
|---|---|---|
| 80 | 2 | 1 |
| 81 | 2 | 3 |
| 82 | 4 | 2 |
| 83 | 3 | 2 |
| 84 | 3 | 2 |
| 85 | 4 | 3 |
| 86 | 2 | 2 |
| 91 | 1 | 1 |

Alternative combinations and variations of the examples provided will become apparent based on this disclosure. It is not possible to provide specific examples for all of the many possible combinations and variations of the embodiments described, but such combinations and variations may be claims that eventually issue.

TABLE 4
Assay comparison for commercial kits and example test kits.

| | | | | | | Trinity | Example Test | |
|---|---|---|---|---|---|---|---|---|
| I.D. # | Western | Abbott | Murex | OraSure | MedMira | Uni- | Kits | |
| PRB204 | Blot | Determine^{Tm} | SUDS | OraQuick ® | Reveal ® | Gold^{Tm} | Result | CIV |
| 01 | − | − | − | − | − | − | No | No |
| | | | | | | | test | test |
| 02 | + | + | + | + | + | + | + | 4 |
| 03 | − | − | − | − | − | − | No | No |
| | | | | | | | test | test |
| 04 | + | + | + | + | + | + | + | 3 |
| 05 | + | + | + | + | + | + | + | 4 |
| 06 | + | + | + | + | + | + | + | 4 |
| 07 | + | + | + | + | + | + | + | 4 |
| 08 | + | + | + | + | + | + | + | 3 |
| 09 | − | − | − | − | − | − | No | No |
| | | | | | | | test | test |
| 010 | Ind. | + | − | − | − | − | + | 1 |
| 011 | + | + | + | + | + | + | + | 4 |
| 012 | + | + | + | + | + | + | + | 4 |
| 013 | Ind. | + | + | +/− | + | − | + | 2 |
| 014 | + | + | + | + | + | + | + | 4 |
| 015 | + | + | + | + | + | + | + | 4 |
| 016 | + | + | + | + | + | + | + | 3 |
| 017 | + | + | + | + | + | + | + | 3 |
| 018 | Ind. | + | − | +/− | + | − | + | 2 |
| 019 | + | + | + | + | + | − | + | 2 |
| 020 | + | + | + | + | + | + | + | 4 |
| 021 | + | + | + | + | + | − | + | 4 |
| 022 | + | + | + | + | + | − | + | 4 |
| 023 | − | − | − | − | − | − | No | No |
| | | | | | | | test | test |
| 024 | Ind. | + | − | − | − | − | − | 0 |
| 025 | Ind. | + | − | + | − | + | − | 0 |

TABLE 5
Assay comparison for commercial kits and example
test kits (band patterns)

| I.D. # PRB204 | RL37 - Band Pattern |
|---|---|
| 01 | No bands |
| 02 | 18, 24, 31, 41, 55, 51, 65, 120, 160 |
| 03 | No bands |
| 04 | 18, 24, 31, 41, 55, 51, 65, 120, 160 |
| 05 | 24, 31, 41, 51, 55, 65, 120, 160 |
| 06 | 18, 24, 31, 41, 55, 51, 65, 120, 160 |
| 07 | 18, 24, 31, 41, 55, 51, 65, 120, 160 |
| 08 | f18, 24, 31, 41, 51, 55, 120, 160 |
| 09 | No bands |
| 10 | 24, 51, 55, f160 |
| 11 | 18, 24, 31, 41, 55, 51, 65, 120, 160 |
| 12 | 18, 24, 31, 41, 55, 51, 65, 120, 160 |
| 13 | 24, f51, f55, f160 |
| 14 | 18, 24, 31, 41, 55, 51, 65, 120, 160 |
| 15 | 18, 24, 31, 41, 55, 51, 65, 120, 160 |
| 16 | 24, f41, 51, 55, 160 |
| 17 | 24, 51, 55, f120, 160 |
| 18 | f24, f51, f55, 160 |
| 19 | 24, f31, 51, 55, f120, 160 |
| 20 | 18, 24, 31, 41, 51, 55, 65, 120, 160 |
| 21 | 24, 51, 55, 160 |
| 22 | 24, 31, 41, 51, 55, 65, 120, 160 |
| 23 | No bands |
| 24 | 24, f51, f55, f160 |
| 25 | f51, f55 |

What is claimed is:

1. A rapid test kit for detection of a disease using a bodily fluid, the kit comprising:
a cellulose filter paper having a detection surface and an opposite surface, the cellulose filter paper selected from cellulose filter papers having a measured flow rate of a phosphate buffered saline from about 0.04 to about 0.4 mL/min/cm², using a modified ASTM standard flow rate measurement;
at least one antigen immobilized on a test portion of the cellulose filter paper;
a staining agent selected such that, when the bodily fluid or a diluent containing the bodily fluid is directly filtered through the cellulose filter paper, the at least one antigen binds with at least one antibody associated with the disease, if the antibody associated with the disease is present at a detectable level within the bodily fluid, such that the staining agent stains at least a portion of the cellulose filter paper;
a destaining buffer selected to remove at least a portion of any non-specific background staining unrelated to binding between the at least one antibody and the least one antigen; and
an absorbent material disposed below under the opposite surface of the cellulose filter paper, such that the absorbent layer absorbs and retains at least a portion of the bodily fluid or a diluent containing the bodily fluid in contact with the cellulose filter paper.

2. The rapid test kit of claim 1, further comprising:
a first housing having an exit aperture and a recess for retaining the cellulose filter paper and the absorbent material; and
a second housing having an inlet aperture and being mounted onto the first housing, such that the cellulose filter paper is secured between the first housing and the second housing such that the at least one antigen is disposed within the inlet aperture.

3. The rapid test kit of claim 1, wherein the cellulose filter paper is selected from cellulose filter papers having a measured flow rate in a range from about 0.04 mL/min/cm² to about 0.2 mL/min/cm².

4. The rapid test kit of claim 3, wherein the range is from about 0.1 mL/min/cm².

5. The rapid test kit of claim 1, wherein the staining agent comprises Protein A.

6. The rapid test kit of claim 4, where the staining agent further comprises a colloidal gold.

7. The rapid test kit of claim 1, further comprising a contrast enhancing agent to increase the contrast with the non-specific background staining, wherein the contrast enhancing agent includes a silver or a silver compound.

8. The rapid test kit of claim 1, wherein the at least one antigen is a viral antigen and is selected from the group of viral antigens capable of detecting antibodies indicative of a viral infection consisting of HIV virus, Hepatitis B virus, Hepatitis C virus, SARS virus and combinations thereof.

9. The rapid test kit of claim 8, wherein the at least one antigen is selected from the group of viral antigens capable of detecting antibodies indicative of a viral infection consisting of an HIV-1 type virus, an HIV-2 type virus, and combinations thereof.

10. A method for detecting a disease using a rapid test kit, comprising:
immobilizing at least one antigen on a cellulose filter paper such that the at least one antigen is concentrated on the cellulose filter paper in a test area and the test area is visible in an inlet window of the rapid test kit;
depositing a sample comprising a diluent and a bodily fluid selected from the group consisting of whole blood, serum and plasma directly on the inlet window such that the sample passes through the cellulose filter paper;
adding a staining agent to the inlet window;
destaining the cellulose filter paper to remove at least a portion of any non-specific background staining; and
determining the presence of binding between the at least one antigen and at least one antibody in the sample in a concentration indicating infection by the disease.

11. The method of claim 10, wherein the step of depositing comprises depositing a sample comprising a diluent and whole blood; and waiting until the whole blood is dispersed in the diluent prior to depositing the sample.

12. The method of claim 10, further comprising: selecting a cellulose filter paper having a flow rate of about 0.04 ml/min/cm² to about 0.4 ml/min/cm²; and the step of depositing deposits at least 5 microliters of the sample and the sample passes through the cellulose filter paper in less than 180 seconds; and the step of determining is performed immediately following the steps of depositing, adding and destaining.

13. The method of claim 10, wherein the step of adding includes adding a Protein A solution coupled to an agent.

14. The method of claim 10, wherein the step of adding includes adding colloidal gold as the agent.

15. The method of claim 10, wherein the step of destaining uses a phosphate buffered saline without delay after the sample passes through the cellulose filter paper.

16. The method of claim 10, wherein the step of depositing includes adding serum to the diluent and the step of depositing passes the sample through the cellulose filter in less than 60 seconds.

17. The method of claim 10, wherein the step of depositing includes adding whole blood to the diluent and the step of depositing passes the sample through the cellulose filter in less than 180 seconds.

18. The method of claim 10, wherein the step of depositing includes adding plasma to the diluent and the step of depositing passes the sample through the cellulose filter in less than 60 seconds.

19. The rapid test kit of claim 1, wherein the absorbent material is a plurality of layers.

20. The rapid test kit of claim 19, wherein the plurality of layers are of a cellulose.

21. A rapid test kit, comprising:
cellulose filter paper having an antibody detection means on a test area of an inlet surface;
the cellulose filter paper having a means for controlling the rate of fluid flow through the cellulose filter paper such that high titer samples have a mean color index value of at least 2 and low titer samples have a mean color index of at least one.
